(19) United States
(12) Patent Application Publication
(10) Pub. No.: US 2024/0173491 A1
(43) Pub. Date: May 30, 2024

(54) INTEGRATED SYSTEMS AND METHODS OF THERAPEUTIC ADMINISTRATION

(71) Applicant: Novastream Therapeutics Inc., Brighton, CO (US)

(72) Inventors: Manuel Ornelas, Brighton, CO (US); Mark Webb, Brighton, CO (US); Jason Novak, Brighton, CO (US)

(21) Appl. No.: 18/553,198

(22) PCT Filed: Mar. 29, 2022
(86) PCT No.: PCT/US22/22378

§ 371 Date: Sep. 29, 2023

Related U.S. Application Data
(60) Provisional application No. 63/167,303, filed on Mar. 29, 2021.

(51) Int. Cl. A61M 15/00; A61M 11/00; G16H 40/67

(52) U.S. Cl. CPC A61M 15/0068; A61M 11/006; A61M 15/0003; G16H 40/67; A61M 2205/276; A61M 2205/3324; A61M 2205/3327; A61M 2205/3334; A61M 2205/3553; A61M 2205/6009; A61M 2205/70

(57) ABSTRACT

The present disclosure provides systems and methods for administering medication to a subject, and convenient means for monitoring the subject's adherence to a treatment protocol (e.g., a recommended or prescribed treatment regimen).

### PRIORITY CLAIM

This application claims priority to U.S. Provisional Patent Application Ser. No. 63/167,303, filed Mar. 29, 2021, the entire contents of which are incorporated herein by reference and relied upon.

### FIELD

The present disclosure provides systems and methods for administering medication to a subject, and convenient means for monitoring the subject's adherence to a treatment protocol (e.g., a recommended or prescribed treatment regimen).

### BACKGROUND

For many therapeutic compounds, an improved route of administration such as inhalation that circumvents liver metabolism, for example, is advantageous. However such methods of administration traditionally require careful monitoring and thus cannot be feasibly applied to patient populations outside the supervision of a medical professional.

For many therapeutics, and subsequent indications, oral routes of administration (or others) are suboptimal or unsatisfactory. In most cases a poor pharmacodynamic profile will preclude a therapy from an indication or in many cases prevent a drug from reaching the commercial market all together. Most therapies have pharmacodynamic profiles that are highly dependent on route of administration. Specific cases where a pharmacodynamic profile may be modulated are most often seen where a novel route of administration can circumvent stomach digestion, liver metabolism, or absorption limits for example. In many cases a high systemic dose must be achieved for therapeutic benefit due to absorption and metabolic factors. Such high systemic doses are often problematic due to liver or kidney burden for example.

### SUMMARY

Systems, devices and methods consistent with the present disclosure solve these disadvantages by providing a monitoring framework comprising a system of integrated sensors, protocols, and methods. By providing a novel inhalation modality and methods framework, physicians and drug companies can now access a broader array of therapeutic profiles for existing drugs as well as the vast library of “shelved drugs” which may be superior in many cases yet lack a suitable delivery and monitoring framework.

Devices, systems, and methods of the present disclosure solve other problems facing physicians pertinent to patient behaviors management. Adherence to a treatment regime is a particularly acute problem without any commercially available engineering controls. Devices, systems, and methods of the present disclosure provide a first-in-kind engineering control to improve treatment adherence while also eliminating improper use. These engineering controls allow for remote, granular real-time treatment monitoring and dose tailoring by medical professionals, an authorized agent, or an AI.

Devices, systems, and methods of the present disclosure solve many of the described problems above as well as many other problems relating to improper drug use. With the implementation of engineering controls and real time treatment monitoring with dose tailoring, a therapy regime can be applied in a more personalized individualized manner thus improving patient outcomes while providing a new stream of adherence and response data never before available.

### BRIEF DESCRIPTION OF THE FIGURES

FIG. 1 is a schematic view of a computer-implemented system for delivering and monitoring a therapeutic regimen consistent with one embodiment of the present disclosure.

FIG. 2 is a schematic view of a software module of a computer-implemented system for delivering and monitoring a therapeutic regimen consistent with one embodiment of the present disclosure.

FIG. 3 is a schematic view of a hardware device of a computer-implemented system for delivering and monitoring a therapeutic regimen consistent with one embodiment of the present disclosure.

FIG. 4 is a perspective view of a hardware device (e.g., nebulizer) configured for use with a computer-implemented system for delivering and monitoring a therapeutic regimen consistent with one embodiment of the present disclosure.

FIG. 5 is an exploded perspective view of the hardware device (e.g., nebulizer) of FIG. 4.

FIG. 6 is an exploded perspective view of the opposite side of the hardware device (e.g., nebulizer) of FIG. 4.

FIG. 7A is a perspective cutaway view of a hardware device (e.g., nebulizer) configured for use with a computer-implemented system for delivering and monitoring a therapeutic regimen consistent with one embodiment of the present disclosure.

FIG. 7B is a perspective view of the bottom of the hardware device (e.g., nebulizer) of FIG. 7A.

FIG. 8 is a perspective view of a manifold configured for incorporation into a hardware device (e.g., nebulizer) configured for use with a computer-implemented system for delivering and monitoring a therapeutic regimen consistent with one embodiment of the present disclosure.

FIG. 9 is a perspective view of a cartridge configured for use with a hardware device (e.g., nebulizer) for use with a computer-implemented system for delivering and monitoring a therapeutic regimen consistent with one embodiment of the present disclosure.

FIG. 10 is a transparent perspective view of the cartridge of FIG. 7A.

FIG. 11 is a perspective view of a cartridge configured for use with a hardware device (e.g., nebulizer) for use with a computer-implemented system for delivering and monitoring a therapeutic regimen consistent with one embodiment of the present disclosure

FIG. 12 is a perspective cutaway view of the hardware device (e.g., nebulizer) of FIG. 7A. including a manifold and cartridge consistent with one embodiment of the present disclosure.

### DETAILED DESCRIPTION

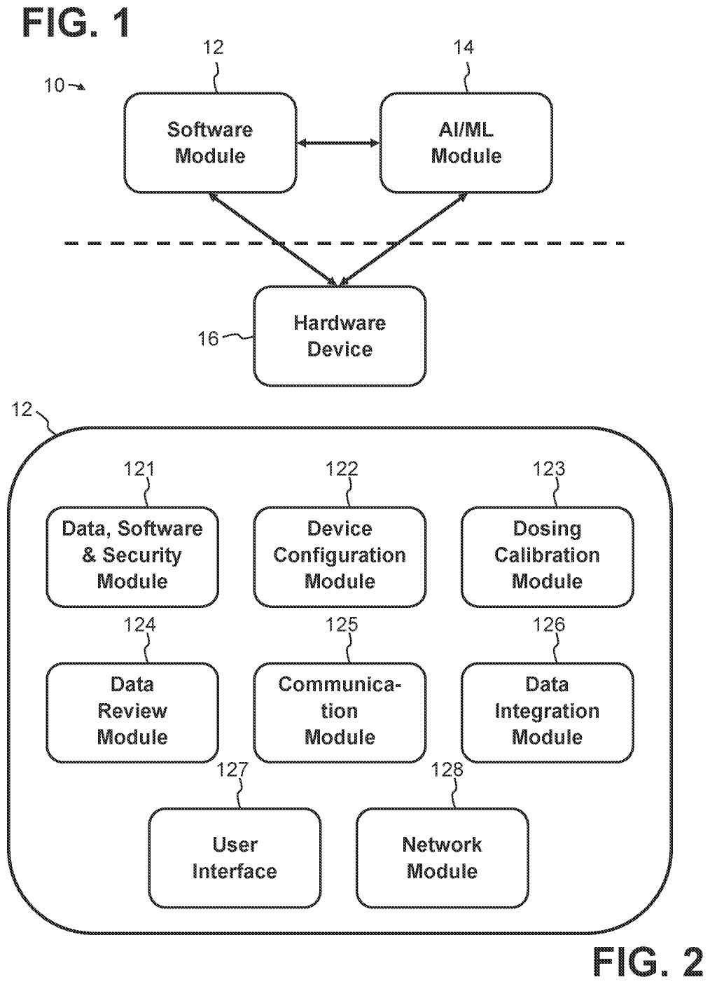

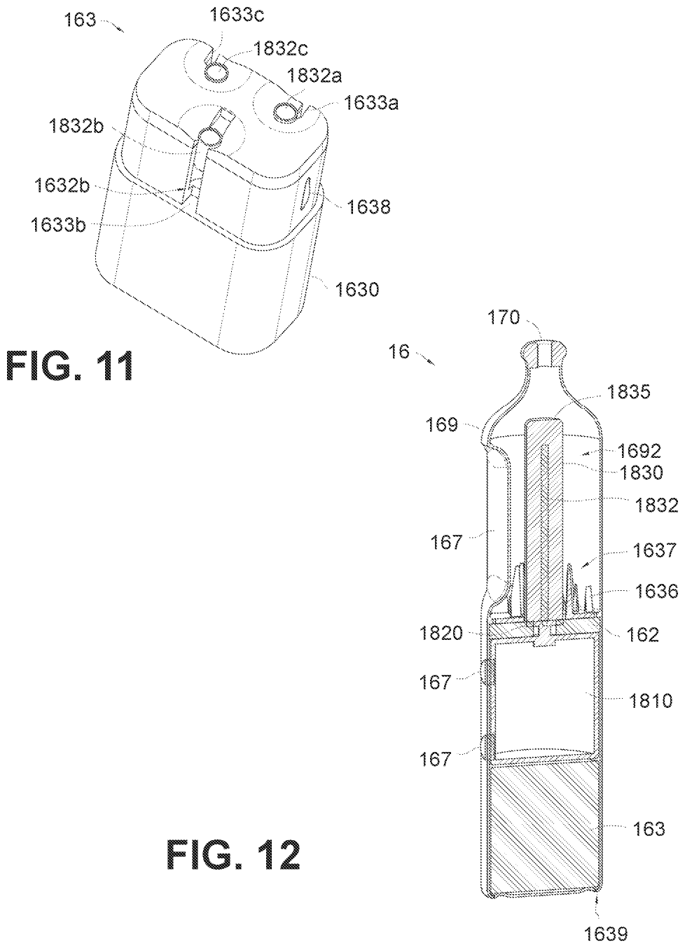

Referring generally to FIGS. 1-12, systems and devices consistent with the present disclosure provide safe, secure, and verifiable means for delivering one or more medicaments to a user.

### 1. Systems

Referring now to FIG. 1, systems 10 of the present disclosure generally include a remote software environment 12 (also referred to as a software module) and one or more associated hardware devices 16 in operable communication with the remote software environment 12. As represented by the dashed line in FIG. 1, the hardware device 16 need not be in close proximity to the remote software module 12, but instead may be in operable communication with the remote software environment (e.g., continuously, contiguously, or intermittently) via a wired, wireless, or cellular communications network (not shown).

In some embodiments, an artificial intelligence or machine learning module 14 is in operable communication with the remote software environment 12 (e.g., is a component of the remote software environment 12). In general, the hardware devices 16 are configured to deliver at least one medicament to one or more users (e.g., patients), while the remote software environment 12 is configured to enable monitoring of, control of, and/or data collection from the associated hardware device(s) 16. When present, the artificial intelligence or machine learning module 14 is configured to receive data from and/or send data to the remote software environment 12, and/or to receive data from and/or send data to with the hardware device(s) 16.

In some embodiments, the system 10 comprises: (a) an integrated drug apparatus (e.g., a hardware device 16); (b) a platform system; (c) a network module and a communication module operably coupled with the platform system, wherein the network module and the communication module communicate data from the integrated drug apparatus; and (d) an inhalation module operably coupled with a controller and a cartridge module, wherein the inhalation module delivers a therapeutic component in a specific dose. In some embodiments, the controller is operable to deliver the specific dose by the inhalation module and record the specific dose data. In some embodiments, the platform system including a one or more modules to initialize and modify the specific dose according to a treatment regimen of a patient, wherein the one or more modules are selected from the group consisting of: a data, software, and device security module 121; a device configuration module 122; a dosing calibration module 123; a data review module 124; a communication module 125; a data integration module 126; a U/I module 127; a logic/compute module 166; a sensor module 168; and combinations thereof. In some embodiments, the the one or more modules are selected from the group consisting of: a data, software, and device security module 121; a device configuration module 122; a dosing calibration module 123; a data review module 124; a communication module 125; a data integration module 126; a U/I module 127; a network module 128; a hardware network module 161; a power module 162; a cartridge module 163; a hardware security module 164; a medicament vehicle module 165; a logic/compute module 166; a hardware input/output module 167; a sensor module 168; and combinations thereof. In some embodiments, the network module is configured to receive, house, and/or transmit a specific dose data, and/or a therapeutic component concentrate data. In some embodiments, the data is standardized for an indication of use of the therapeutic component. In some embodiments, the specific dose data and/or therapeutic component concentrate data is transmitted from a therapeutic component manufacturer. In some embodiments, the network module is configured to receive, house, and/or transmit a dosing regimen. In some embodiments the dosing regimen is generated by an authorized user (e.g., a health care provider) based on patient evaluation, the standardized specific dose data, and/or therapeutic component concentrate data. In some embodiments, the health data and system data is encrypted by a blockchain process. In some embodiments, the network module is configured to receive, house, and/or transmit a patient specific locking encryption. In some embodiments, the network module is configured to generate an associated decryption key by the health care provider. In some embodiments, the integrated drug apparatus is accessible (e.g., only accessible) by satisfying a security mechanism selected from the group consisting of: biometric input mechanism, a fingerprint locking mechanism, a facial recognition locking mechanism, a passcode, an anonymized key, a blockchain key, and combinations thereof. In some embodiments, the encrypted dosing regimen data, the blockchain encrypted dosing regimen data, and/or the patient specific locking encryption, and/or the associated decryption key, is transmitted to a pharmacist. In some embodiments, the pharmacist decrypts and/or reviews the dosing information from the specific dose data and/or therapeutic component concentrate data. In some embodiments, the therapeutic component concentrate or a placebo component is loaded into the integrated drug apparatus or a cartridge module 163 for use with a hardware device 16. In some embodiments, the therapeutic component concentrate loaded into the integrated drug apparatus is loaded with a locking encryption (e.g., based on blockchain) by the pharmacist or health care provider. In some embodiments, the dosing regimen data is loaded with the patient specific locking encryption and the associated decryption key. In some embodiments, the decryption key is used to unlock and activate the integrated drug apparatus by the security mechanisms.

### 2. Remote Software Environments (Modules)

Referring now to FIGS. 1-2, remote software environments 12 consistent with the present disclosure are in operable communication with one or more hardware devices 16. For convenience and clarity, only one hardware device 16 is shown in the representative schematic view of FIG. 1, but the system 10 may include more than one hardware device 16, such as two hardware devices 16, 3 hardware devices 16, 4 hardware devices 16, 5 hardware devices 16, 6 hardware devices 16, 7 hardware devices 16, 8 hardware devices 16, 9 hardware devices 16, 10 hardware devices 16, dozens of hardware devices 16, hundreds of hardware devices 16, or even thousands of hardware devices 16.

In general, the remote software environment 12 is configured to enable convenient monitoring of a user's use of the hardware device 16, to enable placement of use limits on the hardware device 16 conveniently, and/or to remotely collect use data from the hardware device 16.

The remote software environment 12 may include a variety of functional modules. Such modules may be separable software modules or separate software modules in some embodiments. In other embodiments, two or more (e.g., all) of the functional modules may be incorporated into a single body of code.

### (a) Data, Software and Security Module

In some embodiments, the remote software environment 12 includes a data, software and security module 121 configured to prevent misuse of the associated hardware device(s) 16. For example and without limitation, the data, software and security module 121 may be configured to prevent an unauthorized user from causing an associated hardware device 16 to dispense medicament stored in the hardware device 16. For example, the data, software and security module 121 may include instructions that prevent an associated hardware device 16 from dispensing any medicament to a user until the user verifies its identity (e.g., by comparing biometric key data received from the hardware device 16, such as a fingerprint, with stored biometric key data known to be associated with the authorized user of the associated hardware device 16). In such embodiments, the incidence rate of medicament misuse or abuse is reduced or even minimized because the associated hardware device 16 may only enable dispensation of the medicament when the authorized user (e.g., patient associated with the treatment regimen including that medicament) is in close proximity to the hardware device 16.

Alternatively or in addition, the data, security and software module 121 may include instructions that prevent entry or modification of a treatment regimen for an associated hardware device 16 unless login credentials associated with an authorized clinician (e.g., doctor, physician's assistant, pharmacist, etc.) are first provided. In some embodiments, the data, software and security module 121 prevents entry or modification of a treatment regimen associated with a specific class of medicaments (e.g., Schedule II or Schedule III controlled substances) unless login credentials associated with an authorized clinician having proper regulatory authority for that specific class of medicaments is first provided. In some embodiments, the data, software and security module 121 enables read-only access to data associated with an associated hardware device 16 for those with read-only access credentials (e.g., the user, a caretaker of the user, a regulatory agency, an auditor, healthcare insurance staff, etc.), but does not enable such read-only authorized users to enter or modify a treatment regimen associated with a hardware device 16. In some embodiments, the data, software and security module includes instructions to record access to the system 10 by an authorized user to a blockchain ledger. In some embodiments, the data, software and security module includes instructions to record access to the system 10 by an authorized user and a summary of the authorized user's activities in the system 10 (e.g., entry of a new treatment regimen, modification of a treatment regimen, etc.) to a blockchain ledger.

### (b) Device Configuration Module

In some embodiments, the remote software environment 12 includes (e.g., further includes) a device configuration module 122. When present, the device configuration module 122 is configured to enable a clinician to set, alter, or delete a condition (e.g., user limit) associated with an associated hardware device 16. Such conditions may be established, altered, or deleted to improve (e.g., optimize) a user's (e.g., patient's) adherence to the treatment regimen. For example and without limitation, the device configuration module 122 may in some embodiments be configured to enable an authorized clinician to enter a treatment regimen associated with a medicament that enables an associated hardware device 16 to dispense the medicament not more than once every 12 hours. In such embodiments, the associated hardware device 16 will not dispense a second bolus of the medicament until at least 12 hours has elapsed since a previous dispensation of a bolus of the medicament, even if the user attempts to activate the hardware device 16 (e.g., by depressing an input button 167).

In some embodiments, the device configuration module 122 may be configured to enable an authorized clinician to establish one or more conditional limits on use of the hardware device 16. For example and without limitation, the device configuration module 122 may in some embodiments enable an authorized clinician to prevent the hardware device 16 from dispensing a medicament to the user unless the user confirms that a meal has been consumed within a predetermined amount of time (e.g., within 1 hour, within 2 hours, within 3 hours, etc.). In some embodiments, the device configuration module 122 may, upon activation by the user, send a request to the user to confirm that a meal (or other condition precedent) has been satisfied before enabling the hardware device 16 to dispense the medicament to the user.

In some embodiments, the device configuration module 122 is configured to send an alert (e.g., by SMS or other text-based protocol) to an authorized clinician, to the user, or to a third party (e.g., caretaker of the user) if the user attempts to deviate (e.g., substantially deviate) from the treatment regimen associated with the user's hardware device 16. For example and without limitation, the device configuration module 122 may be configured to send an alert when the user attempts to activate the hardware device 16 to dispense a medicament substantially more frequently than prescribed (e.g., as encoded in the treatment regimen) and/or substantially less frequently than prescribed (e.g., as encoded in the treatment regimen). In such embodiments, the alerts may signify a change (e.g., critical change) in the user's health status, such as incapacitation, a worsening of symptoms, a progression of a disease state, or an improvement in symptoms and/or in disease state that might prompt the authorized clinician to contact the user directly for an evaluation or to modify the treatment regimen of the data, software and security module 121 for that user/hardware device 16. In some embodiments, the device configuration module 122 is configured to send an alert (e.g., by SMS or other text-based protocol) to the user, for example as a reminder to dispense the medicament from the hardware device 16 in order to remain adherent (e.g., substantially adherent) to the treatment regimen.

In some embodiments, the device configuration module 122 is configured to enable an authorized clinician (e.g., doctor or physician's assistant) to modify a treatment regimen as the user's (patient's) adherence and/or health status changes. For example, the device configuration module 122 may be configured to enable an authorized clinician to increase a medicament dosage level if the user (patient) gains a significant amount of body mass, or if the user's (patient's) clinical outcomes are not being achieved (e.g., not being achieved at a desired rate).

In some embodiments, the device configuration module 122 may be configured to enable a pharmacist to modify a treatment regimen, or to prompt an authorized clinician to modify the treatment regimen if, for example, the medicament prescribed by the authorized clinician is not available or is substituted by the pharmacist for a different medicament.

In some embodiments, the device configuration module 122 may be configured to enable an authorized user (e.g., clinician, patient, caretaker, pharmacist, regulatory agency, etc.) to specify what data associated with the user's dispensation of the medicament the hardware device 16 collects, and where that collected data is stored or shared. In some embodiments, the device configuration module 122 is configured to enable transmission of hardware device 16 use data only without any patient identifying information.

### (c) Dosing Calibration Module

In some embodiments, the remote software environment 12 includes (e.g., further includes) a dosing calibration module 123. When present, the dosing calibration module 123 is configured to enable an authorized clinician to select a medicament for administration to the user via the hardware device 16, and optionally to enable an authorized clinician to establish a customized initial treatment regimen for the user to follow in dispensing the medicament from the hardware device 16. The customized initial treatment regimen may include any one or more of:
a number of prescribed doses of the medicament (e.g., maximum number of doses) to be dispensed to the user via the hardware device 16;
an amount of medication to be dispensed via the hardware device 16 per dose; and
one or more user-controlled configurations that enable the user to specify (e.g., establish or modify) preferences for dispensing the medicament from the hardware device 16, for example based on user inputs such as the user's typical bedtime, wake up time, meal times, fasting times, etc.

In some embodiments, the dosing calibration module 123 is configured to enable an authorized clinician to receive recommendations (e.g., from a physician's assistant) for incorporation into the treatment regimen. The authorized clinician may be prompted by the dosing calibration module 123 to review and confirm or deny such recommendations.

In some embodiments, the dosing calibration module 123 is configured to enable an authorized clinician to modify the treatment regimen associated with the medicament to be delivered by the hardware device 16, for example in response to a change in the user's health status, diagnosis, and/or physical characteristics (e.g., body mass or age).

In some embodiments, the dosing calibration module 123 is configured to enable a pharmacist to fill a prescription by, for example, coupling a cartridge 163 including the medicament of the treatment regimen with the shell 169 of the hardware device 16 and unlocking the hardware device 16 for use by the user associated with the treatment regimen.

In some embodiments, the dosing calibration module 123 is configured to set (e.g., establish or modify) a duty cycle of the power source 162 of the hardware device 16 supplied to the medicament actuator 1832 (e.g., nebulizer mesh or vaporizer heating element/ultrasonic plate) to deliver the dosage level of the medicament to the user with each activation of the hardware device 16. In some embodiments, the dosing calibration module 123 sets the duty cycle automatically by, for example, causing the power source to provide a calibration duty cycle to the medicament actuator 1832 and determining an amount of the medicament removed from the medicament reservoir 1631a-c (e.g., by one or more sensor measurements as discussed more fully below). In some embodiments, the dosing calibration module 123 repeats this dosing calibration procedure using modified calibration duty cycles until the amount of medicament removed from the medicament reservoir 1631a-c is the same as or is substantially the same as the dosage level of the medicament specified in the treatment regimen.

### (d) Data Review Module

In some embodiments, the remote software environment 12 includes (e.g., further includes) a data review module 124. When present, the data review module 124 is configured to enable an authorized user to view data associated with the user's (patient's) dispensation of medicament(s) from the hardware device 16, data associated with the one or more sensors 168, and/or the therapeutic regimen specified by the authorized clinician. In some embodiments, the data review module 124 is configured to enable a clinician to review usage data generated by the hardware device. In some embodiments, the data review module 124 is configured to enable a user of the hardware device 16 to view data associated with the user's treatment regimen(s) and/or data associated with the user's consumption of the at least one medicament. In some embodiments, the data review module 124 is configured to enable a caretaker of a user of the hardware device 16 to view data associated with the user's treatment regimen(s) and/or data associated with the user's consumption of the at least one medicament. In some embodiments, the data review module 124 is configured to enable a pharmacist to view data associated with a user's treatment regimen. In some embodiments, the data review module 124 is configured to enable a reporting agent or a regulatory agency to view data associated with consumption of the at least one medicament. In some embodiments, the data review module 124 is configured to enable an authorized auditor to view data associated with consumption of the at least one medicament. In some embodiments, the data review module 124 is configured to enable an authorized agent to view data associated with real-time consumption of the at least one medicament by a user of the hardware device 16.

In some embodiments, the data review module 124 is configured to enable an authorized user to generate reports including any data available in the system 10 related to one or more user's dispensation of a medicament via an associated hardware device 16. In some embodiments, the data review module 124 redacts or removes patient-identifying information from such reports if the report is to be made available to unauthorized users or third parties. In some embodiments, the data review module 124 is configured to generate and transmit a report (e.g., without patient-identifying information) including information associated with one or more user's dispensation of a medicament via the hardware device(s) 16 to a third party on a predetermined schedule or interval.

In some embodiments, the data review module 124 is configured to enable an authorized clinician to review, monitor, and report on any data associated with the user's dispensation of the medicament via the hardware device 16.

In some embodiments, the data review module 124 is configured to enable the user (patient) to view the treatment regimen(s) provided by an authorized clinician, and/or medicament consumption data generated by the user's dispensation of the medicament via the hardware device 16. In some embodiments, the data review module 124 enables the user to provide feedback on the treatment regimen to the authorized clinician.

In some embodiments, the data review module 124 is configured to enable a user caretaker (e.g., parent, guardian, or individual with medical power of attorney) to view the treatment regimen(s) provided by an authorized clinician, and/or medicament consumption data generated by the dispensation of the medicament to the user via the hardware device 16. In some embodiments, the data review module 124 enables the caretaker to provide feedback on the treatment regimen to the authorized clinician.

In some embodiments, the data review module 124 is configured to enable a pharmacist to view the treatment regimen(s) provided by an authorized clinician, and/or medicament consumption data generated by the user's dispensation of the medicament via the hardware device 16.

In some embodiments, the data review module 124 is configured to enable a regulatory agent to view treatment regimen(s) provided by authorized clinicians, and/or medicament consumption data generated by users' dispensation of the medicament via the hardware device 16, for example to ensure that the system 10 is operating within required guidelines and/or that the authorized clinicians are properly authorizing dispensation of the medicament to users via the hardware devices 16.

In some embodiments, the data review module 124 is configured to enable an authorized system user to view medicament consumption data in real-time or near-real-time, for example to monitor a user's adherence to or deviation from the treatment regimen.

In some embodiments, the data review module 124 is configured to anonymize patient data (e.g., remove patient-identifying information from data stored by the hardware device 16 and/or by the system 10.

In some embodiments, the data review module 124 is configured to aggregate data stored by multiple hardware devices 16 associated with the system 10, and/or data stored by the system 10 corresponding to multiple hardware devices 16 associated (e.g., previously associated and/or presently associated) with the system 10.

In some embodiments, the data review module 124 is configured to encrypt data stored by one or more hardware devices 16 associated with the system 10, and/or data stored by the system 10 corresponding to one or more hardware devices 16 associated (e.g., previously associated and/or presently associated) with the system 10.

In some embodiments, the data review module 124 is configured to decrypt data stored by one or more hardware devices 16 associated with the system 10, and/or data stored by the system 10 corresponding to one or more hardware devices 16 associated (e.g., previously associated and/or presently associated) with the system 10.

In some embodiments, the data review module 124 is configured to convert data stored by one or more hardware devices 16 associated with the system 10, and/or data stored by the system 10 corresponding to one or more hardware devices 16 associated (e.g., previously associated and/or presently associated) with the system 10, to a data file of a different type.

### (e) Communications Module

In some embodiments, the remote software environment 12 includes (e.g., further includes) a communications module 125. When present, the communications module 125 is configured to enable communications between two or more authorized system users, for example via SMS or other text-based protocol and/or via email.

In some embodiments, the communications module 125 is configured to enable an authorized clinician to communicate with the hardware device 16 user (patient) and/or with the user's (patient's) caretaker.

In some embodiments, the communications module 125 is configured to enable an authorized clinician to communicate with another clinician associated with the hardware device 16 user (patient) who may or may not be an authorized system user. For example and without limitation, the communications module 125 in some embodiments may be configured to enable an authorized clinician affiliated with the treatment regimen (e.g., an endocrinologist) to send and receive communications with another clinician that is not affiliated with the treatment regimen but may benefit from receiving data related to the user's consumption of the medicament (e.g., a dietician).

In some embodiments, the communications module 125 is configured to enable an authorized clinician to communicate with a third party, such as a pharmacist, about the treatment regimen. For example and without limitation, the communications module 125 in some embodiments may enable the authorized clinician who establishes the treatment regimen to receive a request from a pharmacist to substitute the medicament of the treatment regimen with an alternative medicament that is known to present fewer drug interaction risks with another prescription related to the hardware device 16 user (patient).

In some embodiments, the communications module 125 is configured to enable an authorized doctor to communicate with a physician assistant about the hardware device 16 user's treatment regimen, health status, adherence to or deviation from the treatment regimen, etc. In some embodiments, the communications module 125 is configured to enable any authorized system user to communicate with any other authorized system user. In some embodiments, the communications module 125 is configured to prevent communications between authorized system users unless a system administrator has authorized communications between the two authorized system users.

### (f) Data Integration Module

In some embodiments, the remote software environment 12 includes (e.g., further includes) a data integration module 126. When present, the data integration module 126 is configured to receive or store data related to the hardware device 16 user's medical information (e.g., diagnoses, vital signs, known allergies, etc.). In some embodiments, the data integration module 126 is configured to receive and/or store medical information related to the user (patient) that is not generated by the hardware device 16. For example and without limitation, such medical information may include the user's height, body mass (weight), age, gender, known health conditions, previous diagnoses, previous therapeutic regimens, known allergies or sensitivities, vital signs, and the like.

### (g) User Interface Module

In some embodiments, the remote software environment 12 includes (e.g., further includes) a user interface module 127. When present, the user interface module 127 is configured to enable an authorized user to enter login credentials, navigate features of the modules 121-126 of the remote software environment 12, view data stored by the system 10, and generate reports related to the user's/users' dispensation of the medicament via the hardware device(s) 16.

### (h) Network Module

In some embodiments, the remote software environment 12 includes (e.g., further includes) a network module 128. When present, the network module 128 is configured to enable data transfer between the remote software environment 12, the hardware device(s) 16, and the artificial intelligence or machine learning module 14, when present.

In some embodiments, the network module 128 is configured to control a wired, wireless, and/or cellular transducer to affect transmission of data to and from the remote software environment 12.

In some embodiments, the network module 128 is configured to receive data from a second medical device (not shown). For example and without limitation, the network module 128 in some embodiments may be configured to receive health-related data (e.g., treatment data, treatment regimen data, treatment outcomes data, etc.) and/or metadata (e.g., dates, times, and durations of use) from a device that does not deliver the one or more medicaments to the user (patient). Such data may be useful for developing a greater understanding of the user's activities, other unrelated health conditions, lifestyle, etc. Some non-limiting examples of the second medical device include blood glucose monitors, implantable gastric impulse generators, pulse and blood oxygen sensors, CPAP equipment, blood pressure sensors (e.g., sphygmomanometers), a fitness monitoring watch or band (e.g., FitBit devices), brain activity sensors, and the like.

In some embodiments, the network module 128 is configured to receive data from the hardware device 16 related to the user's dispensation of the medicament from the hardware device 16. In some embodiments, the network module 128 is configured to cause the system 10 to store data related to the user's dispensation of the medicament from the hardware device 16 in memory associated with the system 10.

In some embodiments, the network module 128 is configured to receive data from an authorized clinician's device (not shown) related to the treatment regimen, such as an initial treatment regimen, modifications to the treatment regimen, communications to be transmitted to another authorized system user, communications to be transmitted to the user (patient), etc. In some embodiments, the network module 128 is configured to cause the system 10 to store data provided by the authorized clinician in memory associated with the system 10. In some embodiments, the data received is encrypted; in such embodiments, the network module 128 may be configured to pass the received data to a data review module for decryption. In other embodiments, the network module 128 may be configured to decrypt the data. In some embodiments, the data includes information related to the medicament specified in the treatment regimen, such as the medicament identity, the desired administration dose, and/or the desired administration frequency.

In some embodiments, the network module 128 is configured to receive data from an authorized third party's device (not shown) related to the treatment regimen. For example and without limitation, in some embodiments the network module 128 may be configured to receive an inquire from a pharmacist related to the medicament, the medicament dosage, etc. specified in a treatment regimen provided by an authorized clinician. In some embodiments, the network module 128 is configured to cause the system 10 to store data provided by the authorized third party in memory associated with the system 10. In some embodiments, the data received is encrypted; in such embodiments, the network module 128 may be configured to pass the received data to a data review module for decryption. In other embodiments, the network module 128 may be configured to decrypt the data. In some embodiments, the data includes information related to the medicament specified in the treatment regimen, such as the medicament identity, information about the medicament's stability (e.g., shelf life), and/or an amount of the desired medicament available from the third party (e.g., pharmacist).

In some embodiments, the network module 128 is configured to send to or receive data from an authorized non-clinical third party's device (not shown) related to the user's (patient's) dispensation of the medicament via the hardware device 16. For example and without limitation, in some embodiments the network module 128 may be configured to transmit data (e.g., without patient-identifying information) related to one or more user's dispensation of a Class II or Class III controlled substance via the hardware device(s) 16 to a regulatory agency, an oversight body, or an auditor. In some embodiments, the network module 128 is configured to cause the system 10 to store data provided by the authorized third party in memory associated with the system 10.

### 3. Hardware Devices

The present disclosure provides hardware devices 16 suitable for use with a remote software environment 12 to reduce the risk of misuse of one or more medicaments by a user without requiring the user to be closely monitored by a clinician at each dosage of the medicament. Such devices are particularly useful for safely, securely, and verifiably dispensing medicaments prone to misuse or abuse, such as Schedule II and/or Schedule III drugs (e.g., fentanyl, hydromorphone, meperidine, methadone, morphine, oxycodone, fentanyl, dextroamphetamine, methylphenidate, methamphetamine, pentobarbital, secobarbital, benzphetamine, ketamine, phendimetrazine, and anabolic steroids).

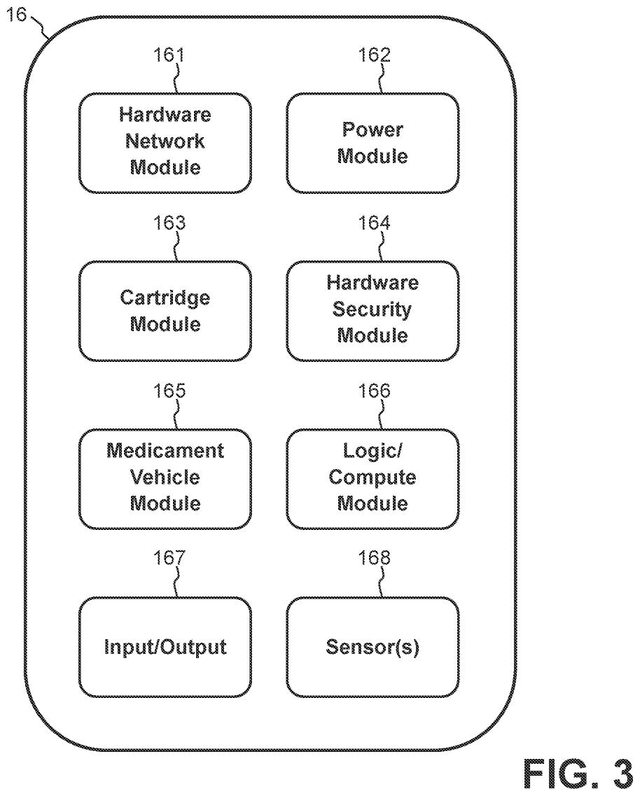

Referring now to FIG. 3, hardware devices 16 consistent with the present disclosure generally include means for securely storing one or more medicaments (e.g., a cartridge module 163), means for dispensing a desired amount of the one or more medicaments (e.g., medicament vehicle module 165 and/or power module 162), means for controlling or limiting dispensation of the medicament(s) from the storage means (e.g., logic/compute module 166, hardware security module 164, and/or hardware network module 161), user inputs/outputs 167, and optionally one or more sensors 168.

In some embodiments, the hardware device 16 includes a storage/mixing chamber 1691 configured to receive a cartridge 163, and a nebulizing chamber 1692 disposed between the storage/mixing chamber 1691 and the exit port 170 and configured to dilute concentrated medicament with a diluent (e.g., air) before the diluted medicament exits the exit port 170.

In some embodiments, the nebulizing chamber 1692 includes a mixing zone 1637 configured to enhance mixing of the diluent (e.g., air) and the concentrated medicament emanating from the storage/mixing chamber 1691. In some embodiments, the mixing zone 1697 includes one or more fins 1636 configured to add a turbulence to the diluent as the diluent (e.g., air) enters the nebulizing chamber 1692. In some embodiments, each fin 1636 is generally helical in shape.

### (a) Hardware Network Module

In some embodiments, the hardware device 16 includes a hardware network module 161. When present, the hardware network module 161 is configured to enable two-way data communication between the hardware device 16 and the remote software environment 12. In some embodiments, the hardware network module 161 is configured to control a wired, wireless, and/or cellular transducer to affect transmission of data to and from the remote software environment 12. In some embodiments, the hardware network module 161 is configured to transmit data to and from the remote software environment 12 via an intermediate device, such as a smartphone or computing device (not shown).

In some embodiments, the hardware network module 161 is configured to receive data from the remote software environment 12 related to restrictions placed by an authorized clinician on the user's ability to dispense the medicament from the hardware device 16.

### (b) Power Module

In some embodiments, the hardware device 16 includes (e.g., further includes) a power module 162. When present, the power module 162 is configured to provide electrical power to components of the hardware device 16 such as, for example, the hardware network module 161, the logic/compute module 166, and the sensor(s) 168. In some embodiments, the power module 162 is configured to provide power to a nebulizer mesh (e.g., piezo element) or vaporizer heating element/ultrasonic plate in the form of a pulse cycle. In some embodiments, the power module 162 is configured to provide a pulse cycle that can be adjusted or varied by the logic/compute module 166 to provide different amounts of nebulized medicament in a unit dose.

In some embodiments, the power module 162 includes a battery, such as a rechargeable battery.

### (c) Cartridge Modules

In some embodiments, the hardware device 16 includes (e.g., further includes) a cartridge module 163. When present, the cartridge module 163 is configured to securely store one or more medicaments to be delivered to the user (patient) by the hardware device 16. In some embodiments, the cartridge module 163 is removeable from the shell 169 of the hardware device 16. In other embodiments, the cartridge module 163 is not removeable from the shell 169 of the hardware device 16.

In some embodiments, the cartridge module 163 includes a medicament actuator 1832, such as a nebulizer mesh or vaporizer heating element/ultrasonic plate element when the hardware device 16 is a nebulizer. In other embodiments, the medicament actuator 1832 is not a part of the cartridge module 163.

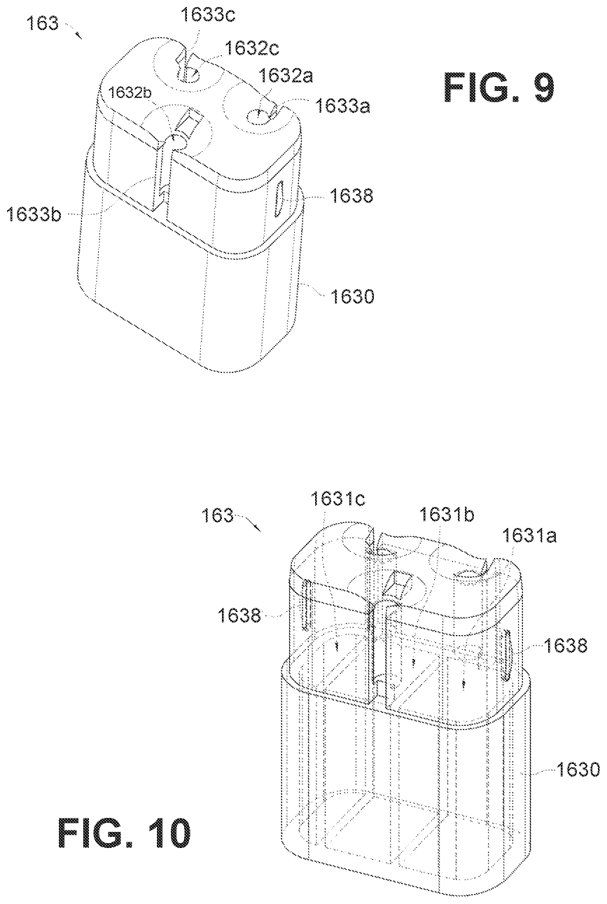

Referring now specifically to FIGS. 9-10, the cartridge module 163 in some embodiments includes a housing 1630 configured to mate (e.g., reversibly mate) with the housing 169 of the hardware device 16. In embodiments wherein the housing is configured to reversibly mate with the housing 169 of the hardware device 16, the cartridge housing 1630 may include one or more securing components 1638, such as latches, detents, springs, protrusions, etc. to enhance purchase of the cartridge housing 1630 with the reusable housing 169 of the hardware device 16.

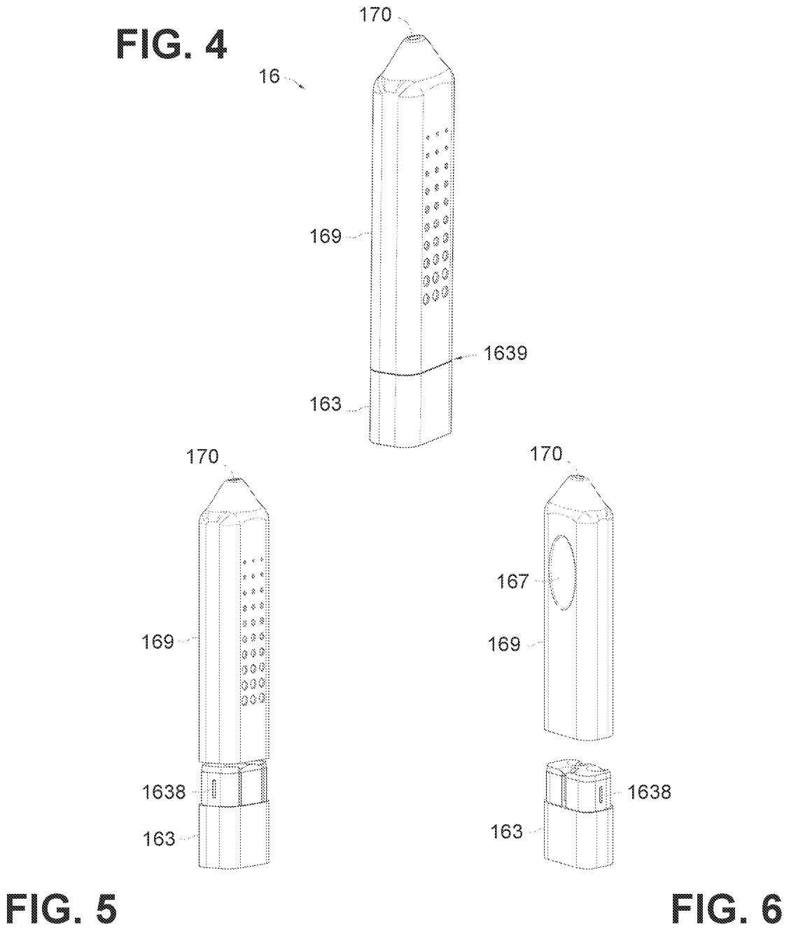

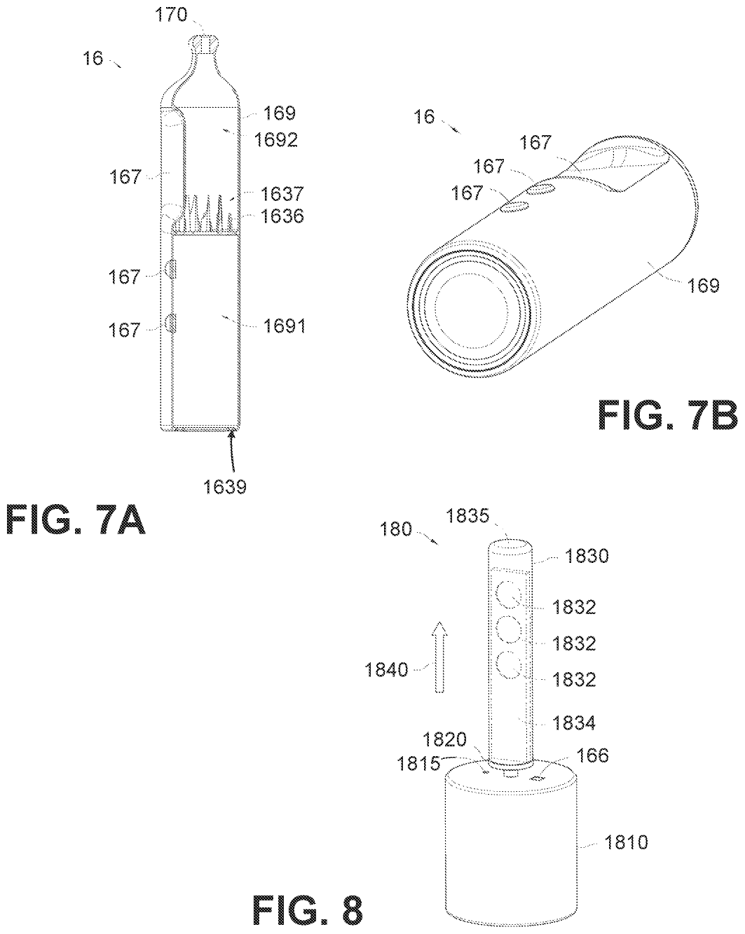

In some embodiments, an air intake gap 1639 is disposed between the housing 1630 of the cartridge module 1630 and the housing 169 of the hardware device 16. When present, the air intake gap 1639 enables air to be drawn into the interior of the hardware device 16 (e.g., into the storage/mixing chamber 1691). In some embodiments, the air intake gap 1639 has an aperture of about 50 μm to about 500 μm, about 100 μm to about 400 μm, or about 200 μm to about 300 μm, for example about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm. In some embodiments, the air intake gap 1639 is disposed on a side wall of the housing 169 (see, e.g., FIGS. 4-6). In other embodiments, the air intake gap 1639 is disposed on a bottom surface of the housing 169 (see, e.g., FIGS. 7A-7B, 12).

In some embodiments, the cartridge 163 further includes a medicament(s) identification indicator (not shown) configured to provide information about the medicament(s) stored in the cartridge module 163. For example and without limitation, the medicament(s) identification indicator may provide information about the identities of the medicaments in the cartridge 163, the concentration of the medicaments in the cartridge 163, the concentration (or range of concentrations) suitable for administration to a human, usage instructions for the hardware device to operate, the amount of each medicament in the cartridge, etc. In some embodiments, the medicament(s) identification indicator includes a chip, a scannable code, an RF transmitter, or similar optical or electronic component configured to be read by an optical scanner or receiver of the hardware device 16.

In some embodiments, the cartridge housing 1630 is formed of a durable chemical-resistant material, such as ABS plastic, reinforced plastic, or similar material to enhance tamper resistance of the cartridge 163, for example to discourage or prevent access to the bulk medicament(s) stored therein. In some embodiments, the cartridge housing 1630 is formed of a metal or metal alloy. In some embodiments, the cartridge housing 1630 is formed of a carbon fiber material to form a carbon fiber pressure canister or similar structure.

The cartridge 163 generally includes one or more medicament reservoirs 1631a-c each configured to store (e.g., securely store) a medicament. In some embodiments, the medicament stored in the reservoir 1631a-c is in a concentrated form, for example at a concentration greater than that desired for administration to the user (patient). In such embodiments, the medicament must be diluted with a suitable diluent or vehicle before emanating from the exit port 170. In other embodiments, the medicament is stored in the reservoir 1631a-c at a concentration equal to or substantially equal to the concentration desired for administration to the user (patient). In such embodiments, the medicament does not need to be diluted with a diluent or vehicle before emanating from the exit port 170.

In some embodiments, the cartridge 163 includes a single medicament reservoir 1631a.

In other embodiments, the cartridge includes two medicament reservoirs 1631a-b. In such embodiments, one reservoir 1631a may include a first medicament to be administered to the user while the second reservoir 1631b may include a second, different medicament to be administered to the user. As used in this context, the term “different medicament” may refer to a medicament having a different active agent(s) than the active agent(s) of the first medicament, or to a medicament having the same active agent(s) than that of the first medicament but at a different concentration and/or in a different vehicle. In some embodiments, the first reservoir 1631a houses a medicament while the second reservoir 1631b houses a medicament vehicle suitable for diluting the medicament stored in the first reservoir 1631a.

In still other embodiments, the cartridge module 163 includes three medicament reservoirs 1631a-c, such as shown representatively in FIGS. 9-10. In such embodiments, each reservoir 1631a-c may store a different medicament, or at least one reservoir may store a medicament vehicle suitable for diluting the medicament(s) stored in the remaining reservoir(s).

The cartridge modules 163 depicted in FIGS. 9-10 are suitable for use in a nebulizing hardware or vaporizing hardware device 16. The cartridge housing 1630 in this representative embodiment includes three reservoirs 1631a-c for securely storing up to three different medicaments and medicament vehicles. Each reservoir 1631a-c is in fluid communication with a feed tube 1632a-c configured to deliver medicament from the reservoir 1631a-c to a manifold 180 (FIG. 8). Feeding of the medicament from the reservoir 1631a-c to the manifold is assisted by airflow vents 1633a-c disposed along the length of each feed tube 1632a-c. In operation, air entering the airflow vents 1633a-c in response to the user's draw on the exit port 170 carries a portion of the medicament from the reservoir 1631a-c upwards toward the manifold 180.

In some embodiments, each reservoir 1631a-c is associated with one medicament actuator 1832 (e.g., nebulizer mesh or vaporizer heating element/ultrasonic plate) capable of being independently energized by the power module 162. In other embodiments, all reservoirs 1631a-c feed medicament via feed tubes 1632a-c to a single medicament actuator 1832 configured to receive power from the power module 162. In some embodiments, each tube 1632a-c is configured to receive a nebulizing mesh element 1632 or vaporizer heating element/ultrasonic plate.

Referring now to FIG. 8, a manifold 180 may be in fluid communication with the cartridge module 163. When present, the manifold 180 may include a storage portion 1810 in which the medicament(s) and a medicament vehicle (e.g., air) are mixed before exiting the exit port 170. In some embodiments, such as when the hardware device 16 is a nebulizer or vaporizer, the storage portion 1810 of the manifold 180 may be in fluid communication with the medicament actuator 1832 (e.g., a nebulizer mesh or vaporizer heating element/ultrasonic plate, such as a piezo element). The medicament actuator 1832 may be in operative communication with the power source 162, for example when the medicament actuator 1832 requires an electrical current to dose the medicament(s) to the user. In some embodiments, the medicament actuator 1832 is supported by an actuator support 1834. For example and without limitation, the manifold 180 shown representatively in FIG. 8 includes a tube 1830 surrounding the medicament actuator 1832. The actuator support 1834 may provide, for example, physical support, electrical insulation, and/or protection from physical damage to the medicament actuator 1832 from physical or vibrational forces.

In some embodiments, the manifold 180 includes a pressure equalizing vent 1815 configured to prevent a pressure differential from persisting between the storage portion 1810 of the manifold 180 and the nebulizing chamber 1692.

In some embodiments, the cartridge module 163 is associated with (e.g., includes) a medicament flow assist (not shown) configured to advance medicament from the one or more reservoirs 1631a-c to the manifold 180. In some embodiments, the medicament flow assist comprises a capillary wick configured to draw at least a portion of the medicament from the reservoir 1631a-c into the manifold 180. In some embodiments, the medicament flow assist comprises a pressurized chamber configured to force at least a portion of the medicament out of the reservoir 1631a-c to the relatively lower pressurized manifold 180. In some embodiments, the medicament flow assist comprises a pump (e.g., micropump) configured to draw at least a portion of the medicament from the reservoir 1631a-c into the manifold 180.

### (d) Hardware Security Module

In some embodiments, the hardware device 16 includes (e.g., further includes) a hardware security module 164. When present, the hardware security module 164 is configured to prevent unauthorized dispensing of the at least one medicament from the hardware device.

In some embodiments, the hardware security module 164 is configured to receive a user input via an input component 167, such as a fingerprint scanner, camera, image recognition ASIC (biometric), encryption ASIC, etc. to verify the user's identity (e.g., in comparison to known user identity information stored in memory of the hardware device 16 or in memory associated with the remote software environment 12).

In some embodiments, the hardware security module 164 is configured to prevent removal of the cartridge module 163 from the shell 169 of the hardware device 16 by the user. For example and without limitation, the hardware security module 164 may in such embodiments be configured to lock the cartridge module 163 to the shell 169 (e.g., via a solenoid or other physical, electrical, or magnetic locking feature) unless and until an input component 167 of the hardware device 16 receives identifying information corresponding to a clinician (e.g., doctor, physician's assistant, or pharmacist) authorized to manipulate (e.g., handle, replace, or dispose of) the medicament(s) stored in the cartridge module 163.

In some embodiments, the hardware security module 164 is configured to prevent unauthorized transfer of or access to data associated with the user (patient) of the hardware device 16. For example and without limitation, the hardware security module 164 may be configured to not display patient-identifying information on an associated screen if an input component 167 does not first receive identifying information corresponding to the user (patient) assigned to that hardware device 16 or corresponding to an authorized clinician associated with the treatment regimen(s) associated with the hardware device 16.

### (e) Medicament Vehicle Module

In some embodiments, the hardware device 16 includes (e.g., further includes) a medicament vehicle module 165. When present, the medicament vehicle module 165 is configured to store and provide a medicament vehicle (e.g., carrier or diluent) to be mixed with the medicament(s) stored in the reservoir(s) 1631a-c.

In some embodiments, the medicament vehicle module 165 is separate from the cartridge module 163.

In other embodiments, the medicament vehicle module 165 is a component of the cartridge module 163. For example and without limitation, in some such embodiments the medicament vehicle module may comprise one or more reservoirs 1631a-c of the cartridge module 163.

The medicament vehicle may comprise any composition suitable for diluting the medicament(s) stored in the cartridge module 163. For example and without limitation, the medicament vehicle may comprise purified water, an aqueous buffer, a preservative, an isotonic agent, one or more emulsifiers, or any two or more of the foregoing for medicaments that are dilutable in aqueous vehicles.

### (f) Logic/Compute Module

In some embodiments, the hardware device 16 includes (e.g., further includes) a logic/compute module 166. When present, the logic/compute module 166 is configured to control user interfaces displayed by the hardware device 16, process user inputs via the input components 167, control communications received by and transmitted from the hardware network module 161, control the power module 162, and control the hardware security module 164, and process signals received from the one or more sensors 168. The logic/compute module 166 may include a memory component configured to store operating instructions for the hardware device 16.

### (g) Input/Output Components

In some embodiments, the hardware device 16 includes (e.g., further includes) one or more input/output components 167. When present, the input/output components 167 are configured to receive user inputs and/or provide an output signal to the user.

In some embodiments, the hardware device 16 includes an identity input component 167 configured to receive information verifying the identity of the user (patient). In some embodiments, receipt of an input by the identity input component 167 causes the logic/compute module 166 to compare the input signal to identity information corresponding to authorized user(s) of the hardware device 16 (e.g., stored locally or stored in memory of the remote software environment) to determine whether the user providing the identity input may activate or access the hardware device 16. In some embodiments, the identity input component is a fingerprint scanner, a touchscreen, a plurality of data input access points, or one or more ancillary devices, such as a smartphone known to be affiliated with an authorized user of the hardware device 16.

In some embodiments, the hardware device 16 includes an input button 167 configured to receive an input signal from the user (patient) seeking to dispense the medicament from the hardware device 16. Upon activation the input button 167 may cause the logic/compute module 166 to query the therapeutic regimen (e.g., stored locally or stored in memory of the remote software environment) to determine whether the medicament may be dispensed to the user consistent with the therapeutic regimen.

In some embodiments, the hardware device 16 includes an output component 167 in operable communication with the hardware network module 161 and configured to transmit and receive data via wired connection, wireless connection, or cellular data connection. In some embodiments, the output component 167 is a networking antenna. In other embodiments, the output component 167 is a wired data port.

### (h) Sensors

In some embodiments, the hardware device 16 includes (e.g., further includes) one or more sensors 168. When present, the sensors 168 are in operable communication with the logic/compute module 166 and are configured to detect one or more operating parameters of the hardware device 16.

In some embodiments, the hardware device 16 includes a vapor flow sensor 166 configured to determine a flow rate and/or concentration of vapor emanating from the exit port 170.

In some embodiments, the hardware device 16 includes a fluid flow sensor configured to determine a flow rate of fluid (e.g., medicament and/or medicament vehicle) from the cartridge module and/or from the medicament vehicle module.

In some embodiments, the hardware device 16 includes a thermometer (e.g., thermocouple) configured to detect a temperature or change in temperature within the hardware device 16.

In some embodiments, the hardware device 16 includes a pressure sensor configured to determine a change in pressure within the hardware device 16, for example in response to a draw provided by the user (patient).

In some embodiments, the hardware device 16 includes an accelerometer configured to determine a change in fluid (e.g., vapor) velocity within the hardware device 16, for example in response to a draw provided by the user (patient).

In some embodiments, the hardware device 16 includes a voltage sensor configured to determine a flow rate of a liquid within the hardware device 16.

In some embodiments, the hardware device 16 includes an optical sensor configured to determine a specific drug load provided by the hardware device 16. In some embodiments, the optical sensor can be used to monitor the level of medicament stored in the cartridge 163, for example to determine an amount of the medicament administered to the user (patient) over a single therapeutic event or over a series of therapeutic events. In some embodiments, the optical sensor is configured to determine the density of the vapor stream emanating from the nebulizer mesh or vaporizer heating element/ultrasonic plate 1820. In some embodiments, the logic/compute module 166 is configured to determine the specific dose and/or total dose of medicament delivered to the user (patient) within a specified time period as a function of at least the determined vapor stream density, the determined vapor flow rate, and optionally medicament cartridge level information.

In some embodiments, the one or more sensors 168 are disposed proximal to the medicament actuator 1832. For example and without limitation, one or more sensors 168 configured to assess the concentration of medicament in a vapor formed by a nebulizer mesh or vaporizer heating element/ultrasonic plate 1832 may be disposed on the actuator support 1834. In some embodiments, one or more sensors 168 are disposed on the interior surface of the housing 169 of the hardware device 16.

### (i) Example Portable Nebulizer Hardware Device

Referring now to FIGS. 4-12, the present disclosure provides a portable nebulizer 16. The portable nebulizer 16 includes a housing 169 defining a storage/mixing chamber 1691 and a nebulizing chamber 1692. The power source (e.g., battery) may be disposed between the storage/mixing chamber 1691 and the nebulizing chamber 1692. The storage/mixing chamber 1691 houses the cartridge 163 and the storage portion 1810 of the manifold 180. An exit port 170 is disposed in the housing 169 in fluid communication with the nebulizing chamber 1692 and generally opposite the storage/mixing chamber 1691.

In some embodiments, the nebulizing chamber 1692 is configured to mix (e.g., dilute) concentrated vapor generated at the manifold 180 with air or other diluent before the diluted vapor exits the exit port 170.

The cartridge 163 includes one, two, or three medicament reservoirs 1631a-c; at least one reservoir 1631a includes a medicament. In some embodiments, the cartridge 163 includes more than one medicament reservoir each configured to store a medicament. If the medicament is in a concentrated form, at least one other reservoir 1631b-c may include a medicament vehicle for diluting the concentrated medicament before administration to the user. In some embodiments, the cartridge 163 is replaceable; in other embodiments the cartridge 163 is not readily removable from the housing 169.

The nebulizing chamber 1692 is in fluid communication with the at least one medicament reservoir 1631a-c and includes a nebulizing mesh-embedded manifold 1830 configured to mix a portion of the medicament with a medicament vehicle and/or with other medicament or diluent before reaching the medicament actuator(s) 1832 embedded in the surface of the nebulizing mesh-embedded manifold 1830. The nebulizing chamber 1692 also includes one or more nebulizing mesh elements 1832 (e.g., piezo element(s)) in electrical communication with the power source 162 via electrical contacts (not shown for clarity), and is configured to vaporize (e.g., nebulize) the medicament to form a medicament vapor that emanates from the top 1835 of the manifold 180. In some embodiments, the one or more medicament actuators 1832 (e.g., nebulizer mesh elements or vaporizer heating elements/ultrasonic elements) is oriented orthogonal or substantially orthogonal (e.g., such that the faces of the actuator(s) are antiparallel, orthogonal, or substantially orthogonal to the direction of travel 1840) to the direction 1840 of travel of the one or more medicament from the storage chamber 1810 to the medicament actuator(s) 1832 to the exit port 1835 of the manifold 180.

A logic/compute module 166 within the nebulizer 16 is configured to enable the nebulizer 16 to receive data from and send data to a remote software environment 12. The remote software environment 12 may include instructions that, when queried by the logic/compute module 166, limit use of the nebulizer (e.g., prevents the power source from energizing the nebulizer mesh or vaporizer heating element/ultrasonic plate 1832) if one or more use conditions are not satisfied.

In some embodiments, the nebulizer 16 additionally includes a hardware network module 161 in operative communication with the logic/compute module 166 and configured to enable two-way data communication between the nebulizer 16 and the remote software module 12.

In some embodiments, the nebulizer 16 additionally includes a hardware security module 164 in operative communication with the logic/compute module 166 and configured to prevent unauthorized dispensing of the medicament from the nebulizer 16.

In some embodiments, the nebulizer 16 additionally includes at least one sensor 168 in operative communication with the logic/compute module 166 and configured to detect at least one characteristic associated with a user's dispensation of the medicament from the nebulizer 16. In some embodiments, the sensor 168 is one or more of: a sensor for assessing a density of vapor emanating from the nebulizing mesh (e.g., a laser particle sensor or ionization type sensor), a flow sensor for assessing an amount of vapor inhaled by a user through the exit port (e.g., a pressure sensor or volumetric flow sensor), a flow sensor for assessing an amount of the medicament withdrawn from the cartridge (e.g., a fluid flow sensor), a position sensor for assessing a change in position of a float or plunger associated with a volume of the medicament, and a pH sensor for assessing a pH level of vapor emanating from the nebulizing mesh.

In some embodiments, the logic/compute module 166 is configured to control power provided to the nebulizer mesh or vaporizer heating element/ultrasonic plate 1832 from the power source in response to calibration data received from the remote software environment 12.

In some embodiments, the logic/compute module 166 is configured to control power provided to the nebulizer mesh or vaporizer heating element/ultrasonic plate 1832 from the power source in response to calibration data received from the remote software environment 12 and in response to information obtained from the one or more sensors 168.

In some embodiments, the logic/compute module 166 is configured to cause the hardware security module 164 to prevent power from flowing from the power source to the nebulizer mesh or vaporizer ultrasonic plate 1820 in response to security information received from the remote software environment 12. In some embodiments, the security information includes information associated with an authorized frequency of medicament dispensation.

In some embodiments, the logic/compute module 166 is configured to receive information obtained from the at least one sensor 168. In some embodiments, the logic/compute module 166 is further configured to transmit the information obtained from the at least one sensor 168 to the remote software environment 12.

### 4. Methods of Use

The present disclosure provides methods of securely dispensing a medicament to a user via a hardware device 16. Methods consistent with the present disclosure offer significant advantages over known methods, especially for dispensing medicaments that are prone to misuse or abuse by a user.

In some embodiments, a method of securely dispensing a medicament to a user comprises specifying, in a remote software environment 12, a treatment regimen for a user comprising one or more initial conditions of authorized dispensation of a medicament for the user; transmitting the treatment regimen information to a hardware device 16, wherein the hardware device 16 comprises: a logic/compute module 166, a power source in operative communication with the logic/compute module 166 and configured to provide power to a medicament delivery actuator 1832, a hardware security module 164 in operative communication with the logic/compute module 166 and configured to prevent unauthorized use of the hardware device 16, a cartridge 163 comprising the medicament at a first concentration, a medicament vehicle module 165 in fluid communication with the cartridge 163 and including a medicament vehicle composition, and an input module 167 in operative communication with the logic/compute module 166 and configured to detect a desired dispensation of the medicament when activated by the user; and permitting dispensation of the medicament to the user if and only if the activation of the input module 167 by the user satisfies all initial conditions of authorized dispensation. In some embodiments, the initial conditions of authorized dispensation comprise one or more of: a medicament name, a medicament dosage level, and a medicament dosage frequency. In some embodiments, the step of permitting dispensation of the medicament comprises enabling, via the logic/compute module 166, power to flow from the power source to the medicament delivery actuator 1832 upon activation of the input module 167 by the user. In some embodiments, the method further comprises specifying, in the remote software environment 12, a second treatment regimen for the user comprising one or more modified conditions of authorized dispensation of the medicament for the user that differs from the initial conditions of authorized dispensation. In some embodiments, the second treatment regimen is specified by a clinician. In some embodiments, the second treatment regimen is specified by an artificial intelligence or machine learning module 14 associated with the remote software environment 12. In some embodiments, the artificial intelligence or machine learning module specifies the second treatment regimen based at least in part on an observed improvement in one or more characteristics of the user after an initial period of time. In some embodiments, the second treatment regimen includes a test condition selected from a list of permitted modified conditions of authorized dispensation. In some embodiments, the list of permitted modified conditions of authorized dispensation includes one or more of: a modified medicament dosage level, a modified medicament dosage frequency, a modified permitted medicament dispensation time of day, and a modified permitted medicament dispensation time relative to a time of consumption by the user of a second medicament or a meal. In some embodiments, the second treatment regimen is specified by the artificial intelligence or machine learning module 14 only after first (a) prompting a clinician to approve or deny the second treatment regimen, and (b) receiving an approval from the clinician in response to the step of prompting the clinician to approve or deny the second treatment regimen. In some embodiments, the medicament delivery actuator 1832 is a nebulizer mesh or vaporizer heating element/ultrasonic plate.

In some embodiments, a method of integrating drug delivery and therapeutic administration consistent with the present disclosure comprises: (a) delivering a therapeutic component in an inhalant suspension in air from an inhalant module; (b) controlling a therapeutic component dose by a controller and recording the therapeutic component dose data; and (c) monitoring the specific dose data to initialize and modify a treatment regimen to a patient. In some embodiments, the method further comprises communicating the therapeutic component dose data and/or the treatment regimen data to a communications module. In some embodiments, the method further comprises communicating data to the hardware device 16 from a software interface to modify a setting of the hardware device 16. In some embodiments, the method further comprises communicating a therapeutic component usage data from the hardware device 16 to a third-party user. In some embodiments, the method further comprises communicating the treatment regimen through a network module to a health care provider. In some embodiments, the method further comprises controlling a dose delivery rate of the therapeutic component dose (optionally, further comprising controlling the therapeutic component dose delivered by the integrated drug apparatus). In some embodiments, the method further comprises maintaining a constant specific dose of the therapeutic component dose by varying the delivered power to the inhalation module. In some embodiments, the method further comprises providing accessibility of the therapeutic component through an identity chip (optionally, wherein the therapeutic component dose is inaccessible when the identity chip is disengaged from the hardware device 16). In some embodiments, the therapeutic component dose is accessible when the identity chip is matched with a digital key. In some embodiments, the method further comprises encoding a control level of the therapeutic component dose by an identity chip. In some embodiments, the method further comprises controlling a fluid flow rate in the integrated drug apparatus. In some embodiments, the method further comprises monitoring a therapeutic component concentration in the integrated drug apparatus. In some embodiments, the method further comprises monitoring a therapeutic component reservoir level in the integrated drug apparatus. In some embodiments, the method further comprises monitoring an internal pressure in the integrated drug apparatus. In some embodiments, the method further comprises monitoring a temperature of the therapeutic component in the integrated drug apparatus. In some embodiments, the method further comprises coupling the integrated drug apparatus with a hardware security module and preventing unauthorized access of the integrated drug apparatus by the hardware security module. In some embodiments, the hardware security module is selected from the group consisting of: a biometric input mechanism, a fingerprint scanner, a camera, an image recognition ASIC unit, an encryption ASIC component, a physical locking mechanism, and a combination thereof. In some embodiments, the method further comprises receiving and/or storing at least one biometric data. In some embodiments, the method further comprises encrypting the at least one biometric data and/or the medical use data. In some embodiments, the method further comprises transferring the encrypted medical use data from a third-party user to an authorized user. In some embodiments, the method further comprises creating a vapor stream from the integrated drug apparatus In some embodiments, the method further comprises coupling a vapor containment integrated component with the inhalant module. In some embodiments, the method further comprises filtering an incoming air stream from the integrated drug apparatus. In some embodiments, the method further comprises monitoring an aerosolized therapeutic component vapor load in the vapor stream. In some embodiments, the method further comprises nebulizing or vaporizing the therapeutic component dose to create the inhalable therapeutic component suspension in air. In some embodiments, the method further comprises compounding the therapeutic component from a concentrate of the therapeutic component and an excipient or compounding a placebo component from a concentrate and an excipient. In some embodiments, the method further comprises generating an inhalable therapeutic component suspension in air using an ultrasonic wave nebulizer, a vibrating mesh, a nozzle, a jet nebulizer, and/or an atomizer. In some embodiments, the method further comprises delivering an aerosolized therapeutic component vapor load (optionally, including an amount of therapeutic component concentrate) within a unit volume of an inhalant vapor stream. In some embodiments, the method further comprises allowing the vapor stream to equilibrate before being inhaled by the patient. In some embodiments, the method further comprises measuring the equilibrated vapor stream and the aerosolized therapeutic component vapor load delivered from the integrated drug apparatus. In some embodiments, the method further comprises tuning the personalized treatment regimen including a feedback control. In some embodiments, the feedback control is selected from the group consisting of: tailoring of the specific dosing parameters through the hardware device 16, tuning a nebulizing rate, tuning a time of use of the integrated drug apparatus, tuning an inhalation volume, tuning a total dose delivered within a given time, tuning the total specific dose in the treatment regimen, and combinations thereof. In some embodiments, the method further comprises operating the integrated drug apparatus through an I/O module. In some embodiments, the method further comprises operably coupling the I/O Module with a at least one ancillary device (optionally, operably coupling the I/O Module with the platform system). In some embodiments, the at least one ancillary device is selected from the group consisting of: smartphones, computers, tablet computers, server system, and combinations thereof. In some embodiments, the method further comprises modifying a hardware setting, an operational setting, and/or the treatment regimen variables by the at least one ancillary device. In some embodiments, the method further comprises operating at least one functional module by at least one sensor integrated with the hardware device 16. In some embodiments, the at least one sensor is selected from the group consisting of: a vapor flow sensor, an optical sensor, a fluid flow sensor, a thermometer, a pressure sensor, an accelerometer, a nephelometer, a voltage sensor, and combinations thereof. In some embodiments, the optical sensor monitors the level of therapeutic component concentrate within a cartridge; the optical sensor monitors the vapor stream density; the vapor flow sensor measures the flow rate of therapeutic component vapor stream that is actively inhaled by the patient; and/or the inhalant module level sensor generates inhalant module level data. In some embodiments, the inhalant module level data, the optical sensor data and/or the vapor stream data determines the specific and/or total dose delivered to a patient within a specified time and/or over the treatment regimen. In some embodiments, the method further comprises collecting data selected from the group consisting a nebulizing rate, a specific dose delivered, a time of use, an inhalation volume, a total dose delivered within a given time, a plurality of security access events, an inhalant module data, an inhalation dynamic data, a therapeutic component saliva concentration, and combinations thereof (optionally, the method further comprising storing the data in a storage system). In some embodiments, the stored data in the storage system is processed by a machine learning module 14 to improve patient behavior, treatment regimen effectiveness, and/or patient outcome via perception and/or application of multidimensional aggregate datasets to make the treatment regimen suggestion to the patient or health care provider. In some embodiments, the method further comprises computing patterns in aggregate datasets, the dataset selected from the group consisting of race, age, weight, geographic location, dose profiles, time of year, and combinations thereof. In some embodiments, the therapeutic component is to treat or prevent the infectious disease including SARS-COV2.

### 5. Computer-Readable Media

The present disclosure provides computer-readable media configured to enable convenient and secure dispensation of one or more medicaments to a user (e.g., patient).

### (a) Computer-Readable Media for Operating a Data, Software and Security Module

In some embodiments, the computer-readable media stores instructions for operating a data, software and security module 121 substantially as described herein. In some embodiments, the computer-readable media stores instructions for a data, software, and device security module coupled with an integrated therapeutic administration system (optionally, or a hardware device 16) for the therapeutic administration to a patient that, when executed by a processor, cause it to encrypt treatment data and/or patient data obtained from the therapeutic administration system or associated hardware device 16.

In some embodiments, the computer-readable media stores instructions that prevent access (e.g., control) of the hardware device 16 unless a biometric key or a verifiable digital signature is provided by the user to the therapeutic administration system or associated hardware device 16.

In some embodiments, the computer-readable media stores instructions that prevent modification of the medicament(s) dose by a user unless the user authenticates his/her identity to the therapeutic administration system or associated hardware device 16, for example through an associated security framework.

In some embodiments, the computer-readable media stores instructions configured to transmit encrypted treatment data and patient data over a network.

In some embodiments, the computer-readable media stores instructions that, when executed, enable a user to: (a) monitor the access of data across the network; and (b) reflect the access event of data in the security framework.

In some embodiments, the computer-readable media stores instructions that, when executed, captures at least one biometric data of a health care provider (e.g., clinician) and stores the at least one biometric data on an associated platform system. In some embodiments, the computer-readable media stores instructions that, when executed, of validates the provided biometric data, and provides access to the therapeutic administration system or hardware device 16 to deliver the therapeutic component dose within a time period (e.g., predetermined time period) from the at least one validated biometric data. In some embodiments, the computer-readable media stores instructions that enable the health care provider (e.g., clinician) to observe data related to the hardware device 16 user's adherence to the prescribed therapeutic regimen.

In some embodiments, the computer-readable media stores instructions that, when executed, enables a user (e.g., authorized user) to log into a platform system operably coupled with the therapeutic administration system or hardware device 16 by a health care provider (e.g., clinician), capture encrypted data, store the encrypted data, and/or communicate the encrypted data through a communications module. In some embodiments, the computer-readable media stores instructions that, when executed, submits the encrypted data for a verifiable digital signature by the patient. In some embodiments, the computer-readable media stores instructions that, when executed, enables the health care provider (e.g., clinician) to write a prescription; send a therapeutic component prescription to a pharmacy; and/or configure the operability of the therapeutic administration system or hardware device 16 according to the therapeutic component prescription. In some embodiments, the computer-readable media stores instructions that, when executed, enables authorization of the therapeutic administration system or hardware device 16 with the encrypted data by the pharmacy. In some embodiments, the computer-readable media stores instructions that, when executed, delivers a therapeutic component prescription to the hardware device 16 use (e.g., patient).

### (b) Computer-Readable Media for Operating a Device Configuration Module

In some embodiments, the computer-readable media stores instructions for operating a device configuration module 122 substantially as described herein. In some embodiments, the computer-readable media stores instructions that, when executed, initialize a set of instructions associated with a patient treatment regimen for the delivery of a therapeutic component dose of one or more medicaments via an associated hardware device 16. In some embodiments, the computer-readable media stores instructions that, when executed, enables configuration of the operability of the therapeutic administration system or associated hardware device 16 by an authorized party, for example to customize a personalized treatment regimen for the user of the hardware device 16 (patient).

In some embodiments, the computer-readable media stores instructions that, when executed, enables establishment of at least one security (lock) setting of the therapeutic administration system or associated hardware device 16 by an authorized user (e.g., clinician or caretaker). In some embodiments, the at least one security (lock) includes a time setting or a time period lock setting. In some embodiments, the computer-readable media stores instructions that, when executed, enable establishment of the at least one lock setting based on patient-generated consumption data or data related to administration of the one or more medicaments associated with a specific time frame.

In some embodiments, the computer-readable media stores instructions that, when executed, prevent an incorrect dosage of drug being delivered by the therapeutic administration system or associated hardware device 16, and/or ensuring a drug adherence by altering the functionality of the therapeutic administration system or hardware device 16 if limits on therapeutic component doses within a time period are reached, a concentration of the therapeutic component dose is reached, or a vapor load of the one or more medicaments is reached.

In some embodiments, the computer-readable media stores instructions that, when executed, enable establishment of at least one alert setting by an authorized user. In some embodiments, the at least one alert setting is configured to alert the hardware device 16 user of a specific therapeutic dosage time or a missed therapeutic dosage time, for example a time defined by the therapeutic regimen.

In some embodiments, the computer-readable media stores instructions that, when executed, enable the at least one alert setting to be configured based at least on the activity of the therapeutic administration system or hardware device 16 within a time period.

In some embodiments, the computer-readable media stores instructions that, when executed, provides an evaluation of the personalized treatment regimen to an authorized user (e.g., clinician). In some embodiments, the computer-readable media stores instructions that, when executed, enables calibration of the treatment regimen in response to the at least one alert setting.

In some embodiments, the computer-readable media stores instructions that, when executed, enables adjustment of the operability of the therapeutic administration system or hardware device 16 during the personalized treatment regimen based at least on patient therapeutic component dosage information available over time.

In some embodiments, the computer-readable media stores instructions that, when executed, enables calibration of the personalized treatment regimen based at least on the patient therapeutic component dosage information available over time.

In some embodiments, the computer-readable media stores instructions that, when executed, enables dispensation of a therapeutic component dose of the at least one medicament when fulfilling a prescription.

In some embodiments, the computer-readable media stores instructions that, when executed, unlocks the therapeutic regimen encoded by the Therapeutic Administration System or hardware device 16 for patient use. In some embodiments, the unlocking is caused after input of at least one biometric data from an authorized user (e.g., a health care provider).

In some embodiments, the computer-readable media stores instructions that, when executed, provides a plurality of data settings and a plurality of configurable settings regarding collected data, transmitted data, and encrypted data. In some embodiments, the plurality of configurable settings determine what data is collected, and/or where collected data is stored.

In some embodiments, the computer-readable media stores instructions that, when executed, enables configuring of the plurality of data settings and the plurality of configurable settings by an authorized user.

In some embodiments, the computer-readable media stores instructions that, when executed, (a) configures the device configuration module settings of the therapeutic administration system or associated hardware device 16 to provide a personalized treatment regimen for the user (patient); (b) loads the therapeutic administration system or hardware device 16 with a therapeutic component and a specific use enabled authorized device configuration; (c) communicates new dosing modifications for the personalized treatment regimen through a software interface by the patient; and/or (d) any combination of two or more of the foregoing.

In some embodiments, the computer-readable media stores instructions that, when executed, (a) captures all patient activity from the Therapeutic Administration System or hardware device 16 by an authorized user (e.g., a health care provider); (b) updates the Therapeutic Administration System or hardware device 16 with new therapeutic component dosing and device configuration settings; (c) tailors the therapeutic component dosing and device configuration settings of the Therapeutic Administration System or hardware device 16 to maintain therapeutic component adherence in a feedback process; and/or (d) any combination of two or more of the foregoing.

### (c) Computer-Readable Media for Operating a Dosing Calibration Module

In some embodiments, the computer-readable media stores instructions for operating a dosing calibration module 123 substantially as described herein. In some embodiments, the computer-readable media stores instructions that, when executed, (a) receives a personalized treatment regimen and a drug input for a hardware device 16 user (e.g., patient); and (b) tailors a therapeutic component dosing and the personalized treatment regimen for the user (patient). In some embodiments, the therapeutic component dosing and the personalized treatment regimen is tailored based at least on the drug information provided by a therapeutic component protocol associated with the therapeutic component.

In some embodiments, the computer-readable media stores instructions that, when executed, provides a number of inhalable units available to a patient according to a configurable timeline within the prescribed treatment regimen.

In some embodiments, the computer-readable media stores instructions that, when executed, ensures a safety range for the prescribed treatment regimen for the patient or suggesting a new therapeutic component and a new personalized treatment regimen.

In some embodiments, the computer-readable media stores instructions that, when executed, configures the personalized treatment regimen of the therapeutic component dosing by an inhalant module. In some embodiments, the computer-readable media stores instructions that, when executed, optimizes the personalized treatment regimen for the user (patient).

In some embodiments, the computer-readable media stores instructions that, when executed, specify an allowable range of therapeutic component dosing and a therapeutic component concentration by a health care provider through a software interface. In some embodiments, the instructions, when executed, modify the personalized treatment regimen by a patient request through the software interface.

In some embodiments, the computer-readable media stores instructions that, when executed, enable an authorized user (e.g., clinician) to adjust the personalized treatment regimen during the course of a treatment regimen. In some embodiments, the instructions, when executed, adjust the therapeutic component dose or therapeutic component concentration based at least on an input related to patient information. In some embodiments, the instructions, when executed, provide a recommendation for the treatment regimen or a modification to the treatment regimen. In some embodiments, the instructions, when executed, enable an authorized user (e.g., clinician) to authorize the modification to the treatment regimen before the recommendation is implemented by the therapeutic administration system or associated hardware device 16.

In some embodiments, the computer-readable media stores instructions that, when executed, authorizes a therapeutic component prescription and configures the therapeutic administration system or hardware device 16 with an inhalant cartridge 163. In some embodiments, the instructions, when executed, unlocks the therapeutic administration system or hardware device 16 when the therapeutic administration system or hardware device 16 is delivered to the patient.

In some embodiments, the computer-readable media stores instructions that, when executed, enable: (a) determination of a device configuration module setting of the Integrated Therapeutic Administration system and/or the hardware device 16 to provide a personalized treatment regimen for the patient; (b) loading the Therapeutic Administration System or hardware device 16 with a therapeutic component and a specific use enabled authorized device configuration; (c) authorization of an updated new therapeutic component dosing regimen by an authorized user (e.g., health care provider) after reviewing the patient consumption data; (d) modification of the therapeutic component dosing regimen to complete a calibration cycle; (e) iterative calibration of the therapeutic component dosing regimen until a personalized treatment regimen adherence is optimized for a patient; and/or (f) any combination of two or more of the foregoing.

### (d) Computer-Readable Media for Operating a Data Review Module

In some embodiments, the computer-readable media stores instructions for operating a data review module 124 substantially as described herein. In some embodiments, the computer-readable media stores instructions that, when executed, (a) generates patient health data from the therapeutic administration system or hardware device 16 and communicates the patient health data to a server system (e.g., a cloud server system); and (b) enables review of the patient health data by an authorized user (e.g., health provider) to facilitate a cloud-based, personalized treatment regimen. In some embodiments, the patient health data includes drug intake, inhalant module activity, patient information data including age, weight, race, sex, genetics, blood type, allergies, or an activity generated on a software interface operably coupled the therapeutic administration system or hardware device 16.

In some embodiments, the computer-readable media stores instructions that, when executed, enables analysis of the patient generated activity through a platform system operably coupled with the server system. In some embodiments, the computer-readable media stores instructions that, when executed, enables tailoring of the personalized treatment regimen based at least on the analyzed patient generated activity. In some embodiments, the computer-readable media stores instructions that, when executed, enables review of the personalized treatment regimen through the software interface. In some embodiments, the computer-readable media stores instructions that, when executed, provides feedback on the personalized treatment regimen, the patient health data, or a time stamped data to the hardware device 16 user (patient) and/or to the authorized user (e.g., clinician or patient's caretaker).

In some embodiments, the computer-readable media stores instructions that, when executed, enables a third party (e.g., pharmacist) to fill a therapeutic component prescription associated with the patient data. In some embodiments, the computer-readable media stores instructions that, when executed, enables a third party (e.g., pharmacist) to fill a therapeutic component prescription associated with the patient data and further enables the third party (e.g., pharmacist) to adjust the personalized treatment regimen.

In some embodiments, the computer-readable media stores instructions that, when executed, reports system usage data according to a regulatory standard of a government agency.

In some embodiments, the computer-readable media stores instructions that, when executed, produces a digital usage date and the system data of the therapeutic administration system or associated hardware device 16 by an authorized user. In some embodiments, the computer-readable media stores instructions that, when executed, protects the digital usage data and system data before being accessible to an unauthorized user. In some embodiments, the computer-readable media stores instructions that, when executed, displays or provides the digital usage data and the system data on demand or on a scheduled basis. In some embodiments, the digital usage data and the system data provide a drug adherence profile associated with one or more users (patients) of associated hardware device(s) 16.

In some embodiments, the computer-readable media stores instructions that, when executed, provides real-time data to an authorized system user for review; adjusts the personalized treatment regimen; and/or provides a drug adherence profile associated with a user of a hardware device 16 associated with the system 10. In some embodiments, the drug adherence profile includes a percentage of days the user (patient) receives a therapeutic component for a period of time, such as from the start of the therapeutic administration until the end of the therapeutic administration.

### (e) Computer-Readable Media for Operating a Communication Module

In some embodiments, the computer-readable media stores instructions for operating a communication module 125 substantially as described herein. In some embodiments, the computer-readable media stores instructions that, when executed: (a) communicates a treatment regimen to a patient through a platform system operably coupled with the hardware device 16; and (b) captures data from the treatment regimen and the hardware device 16 and communicates the functional data to an authorized user (e.g., health provider). In some embodiments, the functional data is selected from the group consisting of: patient health data, therapeutic administration system or hardware device 16 configuration data, treatment regimen data, therapeutic administration system or hardware device 16 treatment regimen event data, and combinations thereof.

In some embodiments, the computer-readable media stores instructions that, when executed, captures the messaging during the course of the treatment regimen of the user (patient).

In some embodiments, the computer-readable media stores instructions that, when executed, adjusts the treatment regimen to modify a drug adherence.

In some embodiments, the computer-readable media stores instructions that, when executed, communicates a prescribed treatment regimen to the authorized user (e.g., health care provider) or to a third-party user. In some embodiments, the computer-readable media stores instructions that, when executed grants authorization to a system user to engage in messaging, communication, and/or data review if the authorization is granted by an administrator of the system 10.

In some embodiments, the computer-readable media stores instructions that, when executed: (a) communicates the therapeutic component dosing or Therapeutic Administration System or hardware device 16 configuration data; (b) communicates real-time data feedback and dialogue between the patient and health care provider; (c) communicates between the health care provider and patient; (d) enables real-time therapeutic component dosing calibration until personalized treatment regimen is optimized for drug adherence; and (e) any combination of two or more of the foregoing.

### (f) Computer-Readable Media for Operating a Data Integration Module

In some embodiments, the computer-readable media stores instructions for operating a data integration module 126 substantially as described herein. In some embodiments, the computer-readable media stores instructions that, when executed: (a) creates a data-centered treatment adherence experience through a platform system operably coupled with the therapeutic administration system or hardware device 16 and a dosing calibration module 123; and (b) integrates data from a third party user selected from the group consisting of: a health care provider, a pharmacist, an associated medical personnel, and combinations thereof.

In some embodiments, the computer-readable media stores instructions that, when executed, loads a patient's data into memory associated with the system 10 for a health care provider to prescribe, monitor, and/or adjust a treatment regimen.

In some embodiments, the computer-readable media stores instructions that, when executed, enables a third party (e.g., pharmacist) to fill a prescription based at least one the patient data elements.

In some embodiments, the computer-readable media stores instructions that, when executed, delivers a personalized treatment regimen to the patient.

In some embodiments, the computer-readable media stores instructions that, when executed, (a) enables input of health and functional treatment regimen data of the patient; (b) integrates the health data of the patient with functional data from the treatment regimen and the therapeutic administration system or hardware device 16; (c) configures the health data and functional treatment regimen data into desired formats in real time; and (d) any combination of two or more of the foregoing.

In some embodiments, the computer-readable media stores instructions that, when executed, (a) enables input of health data and functional treatment regimen data of the patient; (b) integrates the health data of the patient with functional data from the treatment regimen and the therapeutic administration system or hardware device 16; (c) enables review and use of the health data and functional treatment regimen data for the prescription in real time; (d) enables fulfillment of the therapeutic component prescription based at least on the health data and functional treatment regimen data; (e) updates the configuration of the therapeutic administration system or hardware device 16; and (f) any combination of two or more of the foregoing.

### (g) Computer-Readable Media for Operating a Machine Learning Module

In some embodiments, the computer-readable media stores instructions for operating a machine learning module 14 substantially as described herein. In some embodiments, the computer-readable media stores instructions that, when executed, improves patient behavior, treatment regimen effectiveness, and/or patient outcomes based at least on perception and application of multidimensional aggregate datasets to make treatment regimen suggestions. In some embodiments, the computer-readable media stores instructions that, when executed, computes patterns in aggregate datasets including race, age, weight, geographic location, dose profiles, and time of year.

### Examples

Example 1. An Integrated Therapeutic Administration system, comprising:
a. an inhalation module configured and arranged to house a therapeutic component;
b. a controller operable to control delivery of a specific dose of the therapeutic component from the inhalation module (optionally, the controller is operable to record or capture the amount of the specific dosage delivered); and
c. A platform system including a plurality of modules to monitor a treatment regimen of a patient.

Example 2. The system of Example 1, further comprising a network module operably coupled with the platform system (optionally, the network module is operable to communicate the specific dose of therapeutic component data with the platform system and a software interface).

Example 3. The system of Example 2, wherein the software interface is configured to communicate data for the purposes of modifying at least one setting,

Example 4. The system of Example 2, wherein network module is configured to communicate therapeutic component usage data from the system to a user.

Example 5. The system of Example 1, wherein the system is operable to allow communication of a personal treatment regimen from a health care provider to the patient, or vice versa (optionally, the network module of Example 2 is operable to allow communication of a personal treatment regimen from a health care provider to the patient).

Example 6. The system of Example 1, further comprising a Power Module (optionally, the power module is operably coupled to a storage system, the controller, the inhalation module, or a network module).

Example 7. The system of Example 6, wherein the power module supplies power to the integrated Therapeutic Administration system, stores power for the integrated Therapeutic Administration system, or manages power for the integrated Therapeutic Administration system.

Example 8. The system of Example 6, wherein the at least one module is selected from the group consisting of a compute module, an I/O module, a device configuration module, a dosing configuration module, and combinations thereof (optionally, wherein the power module is operably coupled to the at least one module to control a dose delivery rate and/or the specific dose delivered by the integrated Therapeutic Administration system) (optionally, wherein the at least one module varies the delivered power to the inhalation module in order to achieve the desired specific dose).

Example 9. The system of Example 1 wherein the specific dose delivered comprises the total therapeutic component amount per unit volume of inhalable treatment suspension in air.

Example 10. The system of Example 1, further comprising a cartridge module operably coupled with the Integrated Therapeutic Administration system (optionally, the cartridge module includes an identity chip).

Example 11. The system of Example 14, wherein the one or more therapeutic components, concentrates, one or more diluents, or one or more placebo components is inaccessible when the cartridge module is disengaged from the Integrated Therapeutic Administration system.

Example 12. The system of Example 14, wherein the one or more therapeutic components, concentrates, one or more diluents, or one or more placebo components is accessible when the identity chip is matched with a digital key or a biometric key.

Example 13. The system of Example 10, wherein the cartridge module includes an identity chip configured to encode a drug control level of the specific dose delivered of the therapeutic component.

Example 14. The system of Example 10, wherein the cartridge module comprises a plurality of therapeutic component reservoirs containing one or more therapeutic components, concentrates, one or more diluents, or one or more placebo components.

Example 15. The system of Example 10, wherein the cartridge module is operably coupled with at least one sensor (optionally, wherein the at least one sensor is selected from the group consisting of a flow meter to detect fluid flow rates, a biosensor to detect therapeutic component concentrations, an electrical sensor to detect reservoir levels, a pressure sensor, a thermometer, an optical sensor, and/or combinations thereof).

Example 16. The system of Example 1, further comprising a Hardware Security Module.

Example 17. The system of Example 16 wherein the hardware security module prevents unauthorized access to the system.

Example 18. The system of Example 16, wherein the hardware security module is selected from the group consisting of: a biometric scanner to receive biometric input or a biosignature, a fingerprint scanner, a camera, an image recognition ASIC unit, an encryption ASIC component, and a physical locking mechanism.

Example 19. The system of Example 16, wherein the Hardware Security Module is configured and arranged to receive and store at least one biometric data element, encrypts the at least one biometric data, and/or transfers the encrypted biometric data to be received by a user with an authorization key (optionally, the hardware security module is configured to enable alerts to be sent to authorized users regarding device and/or drug usage events).

Example 20. The system of Example 1, wherein the Inhalant Module comprises a vapor stream module or vapor containment integrated component to create a vapor stream, wherein the vapor stream is an inhalable form of the therapeutic component.

Example 21. The system of Example 1, wherein the inhalant module comprises a filtering component to filter an incoming air stream to the inhalant module.

Example 22. The system of Example 1, wherein the inhalant module comprises a metering component that monitors an inhalant therapeutic component suspension in air.

Example 23. The system of Example 1, wherein the inhalant module is a nebulizer or a vaporizer that creates the inhalable drug suspension in air.

Example 24. The system of Example 1, wherein and the inhalant module comprises a therapeutic component concentrate (optionally, wherein the therapeutic component concentrate includes a compounded formulation of the therapeutic component concentrate and an excipient contained within the inhalant module).

Example 25. The system of Example 1, wherein the inhalant module is a nebulizer that generates an inhalable aerosol selected from the group consisting of an ultrasonic wave nebulizer, a vibrating mesh, a nozzle, a jet nebulizer, an atomizer, and combinations thereof.

Example 26. The system of Example 25, wherein the nebulizer delivers an aerosolized therapeutic component vapor load (optionally, the vapor load including an amount of therapeutic component concentrate within a unit volume of the vapor stream).

Example 27. The system of Example 1, wherein the inhalant module is operably coupled with a conduit to allow a vapor stream to equilibrate before being inhaled by the patient.

Example 28. The system of Example 1, wherein the inhalant module comprising a metering component to measure an equilibrated vapor stream and an aerosolized therapeutic component vapor load by a Sensor Module.

Example 29. The system of Example 1, wherein the controller comprises computing hardware selected from the group consisting of a PCB integrated logic/compute chip components or an Application-Specific Integrated Circuit (ASIC).

Example 30. The system of Example 29, wherein the computing hardware allows for a data function comprising a data collection function, a data manipulation function, or a data transfer function.

Example 31. The system of Example 1, wherein the system comprises a Graphical User Interface (GUI) operation component

Example 32. The system of Example 1, wherein the platform system comprises a plurality of interfacing components (e.g., Software UI mechanisms interacting with hardware input—and the AI interactions between both).

Example 33. The system of Example 1, wherein the platform system comprises a plurality of cloud integration components configured for real-time data exchange.

Example 34. The system of Example 1, wherein the platform system comprises a plurality of security features and operation components.

Example 35. The system of Example 1, wherein the platform system comprises a plurality of sensing components (e.g., accelerometers, vapor flow sensors, optical sensors, fluid flow sensors, thermometers, pressure sensors, accelerometers, inhalant module sensors, ionization sensors, resistance sensors, a nephelometer, capacitive sensors, voltage sensors).

Example 36. The system of Example 1, wherein the controller and the platform system further comprises a Treatment Tuning component enabling a feedback control (optionally, wherein the feedback control includes tuning the medicament and diluent usage rates that are inputs to the inhalant module).

Example 37. The system of Example 36, wherein the feedback control includes tailoring of the specific dosing parameters (optionally, the feedback control comprises a plurality of sensors for measuring the specific dose delivered and/or the treatment regimen).

Example 38. The system of Example 36, wherein the feedback control includes tuning a nebulizing rate of the therapeutic component from the inhalant module.

Example 39. The system of Example 36, wherein the feedback control includes tuning a time of use of the inhalant module.

Example 40. The system of Example 36, wherein the feedback control includes tuning an inhalation volume from the inhalant module (optionally, the feedback control comprises at least one module operably coupled with the platform system, wherein the at least one module is selected from the group consisting of: a sensor module, a I/O module, a Data module, and a dosing calibration module).

Example 41. The system of Example 36, wherein the feedback control includes tuning a total dose delivered from the inhalant module within a given time (optionally, wherein the feedback control includes tuning a total dose delivered from the inhalant module within a given time provided by an input from a sensing module).

Example 42. The system of Example 36, wherein the feedback control includes tuning a total therapeutic component dose from the inhalant module in the treatment regimen.

Example 43. The system of Example 1, further comprising an I/O Module operably coupled with the controller.

Example 44. The system of Example 43, wherein the I/O module comprises a plurality of interfacing buttons.

Example 45. The system of Example 43, wherein the I/O module comprises a touchscreen or at least one digital display (optionally, wherein the I/O modules comprises a notification feature) (optionally, wherein the I/O modules comprises a graphical user interface) (optionally, wherein the I/O modules comprises a software interface) (optionally, the notification feature can comprise an indicator light, a vibration feature, and combinations thereof).

Example 46. The system of Example 2, wherein the network module comprises a networking antenna (optionally, the networking antenna is configured and arranged to communicate the therapeutic component specific dose data).

Example 47. The system of Example 43, wherein the I/O module comprises a plurality of data input access points.

Example 48. The system of Example 43, wherein the I/O module comprises a plurality of interfacing elements (e.g., indicator light, a vibration feature, digital display, touch screen, fingerprint scanner, camera, interfacing buttons, dials, microphones, speakers, piezoelectric transducers).

Example 49. The system of Example 43, wherein the I/O Module is operably coupled with more than one ancillary device coupled to the platform system.

Example 50. The system of Example 49, wherein the more than one ancillary devices include a smartphone, a computer, a tablet computer, or a server system.

Example 51. The system of Example 49, wherein the more than one ancillary devices allow for modification of a hardware setting, an operational setting, a security setting, or a treatment regimen variable.

Example 52. The system of Example 1, further comprising a sensor module operably coupled with the controller.

Example 53. The system of Example 52, wherein the sensor module includes a plurality of sensors integrated with the platform system that facilitate operation of the plurality of modules to monitor the treatment regimen of the patient.

Example 54. The system of Example 53, wherein the plurality of sensors are selected from the group consisting of: vapor flow sensors, optical sensors, fluid flow sensors, thermometers, pressure sensors, accelerometers, inhalant module sensors, ionization sensors, resistance sensors, a nephelometer, capacitive sensors, voltage sensors, and combinations thereof.

Example 55. The system of Example 54, wherein the optical sensors monitor a level of the therapeutic component concentrate within the inhalant module.

Example 56. The system of Example 54, wherein the optical sensors monitor a vapor stream density.

Example 57. The system of Example 54, wherein the vapor flow sensors measure a flow rate of therapeutic component vapor stream that is actively inhaled by a patient.

Example 58. The system of Example 54, wherein the inhalant module sensors generate inhalant module event data.

Example 59. The system of Example 54, wherein, depending on the sensor chosen, the optical sensors are configured and arranged to monitor data indicating a level of the therapeutic component concentrate within the inhalant module; the optical sensors are configured and arranged to monitor data indicating the vapor stream density; the vapor flow sensors are configured and arranged to measure data indicating a flow rate of therapeutic component vapor stream that is actively inhaled by a patient; and/or the inhalant module sensors are configured and arranged to generate inhalant module event data (optionally, the optical sensor data and/or the vapor stream data determine the specific dose delivered to a patient within a specified time) (optionally, the optical sensor data and/or the vapor stream data determines the specific and total dose delivered to a patient within a specified time).

Example 60. The system of Example 53, wherein the plurality of sensors collect data elements selected from the group consisting of a Nebulizing rate, a Specific dose delivered, a time of use, an inhalation volume, a total dose delivered within a given time, a plurality of security access events, a therapeutic component delivery inhalant module data, an inhalation dynamic data, a therapeutic component saliva concentration, and combinations thereof (optionally, the plurality of sensors stores the Data in a storage system).

Example 61. The system of Example 60, further comprising a Machine Learning Module operable with a compute module (optionally, wherein the module comprises a set of instructions for processing the data collected by the plurality of sensors for improving patient behavior, improving treatment regimen effectiveness, and/or improving patient outcomes) (optionally, the machine learning module is configured and arranged to process the application of multidimensional aggregate datasets to make treatment regimen suggestions to a user) (optionally, the machine learning module is configured and arranged to compute patterns in aggregate datasets including race, age, weight, geographic location, dose profiles, and/or time of year).

Example 62. The system of Example 1 further comprises a Data, Software, and Device Security Module (optionally, the Data, Software, and Device Security Module includes a set of instructions operably and is coupled to a storage system) (optionally, the Data, Software, and Device Security Module includes an encryption processing system operably coupled to the storage system).

Example 63. The system of Example 62, wherein the Data, Software, and Device Security Module further comprises
a. A biometric capture system to capture at least one biometric data input and store the at least one biometric data in the storage system.

Example 64. The system of Example 62, wherein the Data, Software, and Device Security Module further comprises an authorization system to authorize the patient to access the specific dose once the biometric data is verified.

Example 65. The system of Example 62, the Data, Software, and Device Security Module comprising an encryption processing system configured and arranged to encrypt at least one generated data element and/or at least one data element stored on the storage system.

Example 66. The system of Example 1, wherein therapeutic component is selected from the group consisting of include medicament, drug, diluent, placebo, excipient, and combinations thereof. (optionally, wherein dose is selected from the group consisting of medicament dose, drug dose, diluent dose, excipient dose, and combinations thereof).

Example 67. The system of Example 65, wherein the encryption processing system monitors access to the encrypted data that is verifiable.

Example 68. The system of Example 65, wherein the encryption processing system monitors each time data is accessed.

Example 69. The system of Example 65, wherein the encryption processing system affects an access event on a data chain of custody management.

Example 70. The system of Example 65, wherein the encryption processing system comprises a secure means of authorizing access data that is verifiable.

Example 71. The system of Example 64, wherein the authorization system is configured and arranged to allow the authorization of encrypted data access, delivery of the specific dose, or modification of at least one system setting.

Example 72. The system of Example 64, wherein the authorization system includes a security key is configured and arranged to be transmitted and verified over a network.

Example 73. The system of Example 63, wherein the biometric capture system includes a biometric hardware component is configured and arranged to capture biometric data and store the biometric data within the Platform system.

Example 74. The system of Example 63, further comprising a capture device wherein at least one the biometric data is provided to the Platform system through the capture device (optionally, the capture device is external to the system).

Example 75. The system of Example 64, wherein the biometric capture system is configured and arranged to validate the at least one biometric data to unlock the device and a unit of therapeutic component to be dispensed by the inhalant module.

Example 76. The system of Example 1 further comprises a Device Configuration module comprising a device configuration element configured to be customized for each patient (optionally, through the platform system).

Example 77. The system of Example 76, wherein the device configuration defines a personalized treatment regimen.

Example 78. The system of Example 77, wherein the personalized treatment regimen is customized by a health care provider through a central server system operably coupled to a networking module and the Platform system.

Example 79. The system of Example 77, wherein the system is configured to store the personalized treatment regimen on a storage system (optionally, wherein the system is configured to control a therapeutic component delivery setting) (optionally, wherein the therapeutic component delivery setting allows for the delivery of the therapeutic component for a period of time based on Patient-generated consumption data or according to a prescribed dosing time frame).

Example 80. The system of Example 77, wherein the personalized treatment regimen is customized by a health care provider, and wherein the Device Configuration module is configured and arranged to store a set of instructions for the personalized treatment regimen on a storage system (optionally, whereby the instructions provide an alert to the patient on a specific activity period).

Example 81. The system of Example 77, wherein the personalized treatment regimen is customized by a health care provider and the platform system stores the instructions for the customized personalized treatment regimen on the storage system.

Example 82. The system of Example 81, wherein the personalized treatment regimen includes the device configuration.

Example 83. The system of Example 82, wherein the system operability is adjusted during a period of time of the treatment regimen according to a patient data input that is available during the period of time.

Example 84. The system of Example 77, wherein the personalized treatment regimen is authorized by an authorized party via a security module (optionally, wherein the authorization enables the fulfillment of a therapeutic component prescription).

Example 85. The system of Example 84, wherein the authorized party (a) authorizes the device for use via modification of the device configuration module and (b) applies a transmitted biometric data to authorize the device dosing mechanism for a patient (optionally, wherein the authorized party includes a pharmacist, physician, nurse, or any other health care provider).

Example 86. The system of Example 77, wherein the Device Configuration module comprises at least one data setting.

Example 87. The system of Example 86, wherein the at least one data setting is selected from the group consisting of a device security setting, a collected data setting, a transmitted data setting, an encrypted data setting; a data storage setting, a configured data setting configured by an authorized party (optionally, wherein the authorized party includes a pharmacist, physician, nurse, or any other health care provider).

Example 88. The system of Example 1, further comprising a Dosing Calibration Module comprising instructions to control a variable of the treatment regimen (optionally, the variable can include a drug Selection, a number of prescribed doses, a length of prescription of the therapeutic component, a concentration of therapeutic component per dose, a therapeutic component consumption limit, an integrated Therapeutic Administration system operability limit, and/or a real time data acquisition.)

Example 89. The system of Example 88, wherein the drug selection includes an appropriate treatment regimen based on a therapeutic component information input from a manufacturer, a therapeutic component company, and/or a pharmaceutical company.

Example 90. The system of Example 88, wherein number of prescribed doses comprises the number of specific doses available to a patient according to a configurable timeline within a prescribed treatment regimen.

Example 91. The system of Example 88, wherein the dosing calibration module is configured to modify an amount of therapeutic component per dose to adhere to the personalized treatment regimen prescribed by a health care provider.

Example 92. The system of Example 88, wherein the dosing calibration module is configured to maintain an allowable range of a Patient-led dosing configuration that is specified by a health care provider.

Example 93. The system of Example 92, wherein the dosing calibration module stores a range of therapeutic component dose and concentration instructions for the Patient-led configurations through a I/O module.

Example 94. The system of Example 88, wherein the dosing calibration module is configured to receive dosing configuration instructions from a machine learning module.

Example 95. The system of Example 88, wherein the dosing calibration module comprises a Pharmacist Prescription Authorization sub-module.

Example 96. The system of Example 88, wherein the dosing calibration module comprises a Pharmacist Prescription Authorization sub-module configured to receive an authorization by a Pharmacist.

Example 97. The system of Example 88, wherein the dosing calibration module comprises a Pharmacist Prescription Authorization sub-module comprising a locking mechanism operably coupled to the integrated Therapeutic Administration system to permit authorized therapeutic component dispensing by the patient.

Example 98. The system of Example 1, further comprising a Data Review Module comprising a set of instructions (optionally, the Data Review Module comprising a personalized treatment regimen).

Example 99. The system of Example 98, wherein the data review module generates data related to all system events and/or generates patient health data operably communicable over a network.

Example 100. The system of Example 99, wherein the patient health data is selected from the group consisting of drug intake, inhalation module activity, at least one activity generated by a software interface or an I/O module and combinations thereof (optionally, the data review module is configurable for an automatic customization of the personalized treatment regimen based on patient data) (optionally, patient data comprising age, weight, race, sex, genetics, and/or blood type) (optionally, the data review module is configured to receive or house an instruction encoded into the system for the personalized treatment regimen).

Example 101. The system of Example 98, wherein the data review module comprises a calibration system for adjusting the personalized treatment regimen in response to the patient health data reported through the data review module after an initial specific dose regimen is prescribed.

Example 102. The system of Example 98, wherein the data review module is configured to update the personalized treatment regimen and patient health data (optionally, wherein the update is according to prescription time ranges within the I/O module and/or a feedback system input to a dosing calibration module).

Example 103. The system of Example 98, wherein the data review module comprises an authorized user access point to review the patient health data and the personalized treatment regime (optionally, wherein the data review module enables modification of the personalized treatment regimen).

Example 104. The system of Example 98, wherein the data review module comprises an authorized user access point to review the patient health data and the personalized treatment regimen wherein the data review module facilitates therapeutic component prescription fulfillment.

Example 105. The system of Example 98, wherein the data review module comprises a regulatory reporting system to produce reports according to the regulatory standards of an authorized agency.

Example 106. The system of Example 98, wherein the data review module comprises a data reporting system to produce reports derived from at least one source (optionally, wherein the source is selected from the group consisting of platform system data, I/O module data, dosing regimen data, patient health data, system events, sensor data, and combinations thereof) (optionally, wherein the data review module is configured to generate adherence reports from at least one source selected from the group consisting of platform system data, I/O module data, dosing regimen data, patient health data, system events, sensor data, and combinations thereof).

Example 107. The system of Example 106, wherein the data reporting system encrypts a patient health data and generates a report on a scheduled time period.

Example 108. The system of Example 1, further comprising a Communication Module (optionally, wherein the Communication Module comprises a set of instructions to enable and manage communication between devices and/or authorized individuals/parties).

Example 109. The system of Example 108, further comprising a calibration system to calibrate the personalized treatment regimen and maintain prescription adherence.

Example 110. The system of Example 108, wherein the Communication Module captures a patient health data during a personalized treatment regimen and/or a treatment request.

Example 111. The system of Example 108, wherein the communication system comprises a user.

Example 112. The system of Example 108, wherein the communication system comprises an authorized administrator to authorize communication and data review.

Example 113. The system of Example 1, further comprising a Data Integration Module, (optionally, wherein the data integration module comprises a set of instructions configured to integrate data from a user to enable a data-centered treatment adherence regimen).

Example 114. The system of Example 1, further comprising a Machine learning Module operably coupled with the platform system, (optionally, the ML Modules comprises a set of instructions to improve a patient behavior, a treatment regimen effectiveness, and/or a patient outcome via perception and/or application of multidimensional aggregate datasets to make a treatment regimen suggestion) (optionally, the machine learning module is configured and arranged to compute patterns in an aggregate dataset) (optionally, the aggregated data set can include race, age, weight, geographic location, dose profiles, and/or time of year).

1-17. (canceled)

18: A nebulizer comprising:
a cartridge including at least one reservoir including a medicament, wherein the cartridge is optionally replaceable;
a nebulizing chamber in fluid communication with the at least one reservoir and including:
a manifold configured to mix a portion of the medicament with a diluent, and
a nebulizing mesh configured to vaporize the portion of the medicament;
a power source configured to energize the nebulizing mesh;
an exit port through which the vaporized portion of the medicament exits the nebulizer; and
a logic module in operative communication with a remote software module and configured to limit use of the nebulizer in response to data received from the remote software module.

19: The nebulizer of claim 18 further comprising a hardware network module in operative communication with the logic module and configured to enable two-way data communication between the nebulizer and the remote software module.

20: The nebulizer of claim 18 further comprising a hardware security module in operative communication with the logic module and configured to prevent unauthorized dispensing of the medicament from the nebulizer.

21: The nebulizer of claim 18 further comprising at least one sensor in operative communication with the logic module and configured to detect at least one characteristic associated with a user's dispensation of the medicament from the nebulizer.

22: The nebulizer of claim 21, wherein the at least one sensor includes one or more of: a sensor for assessing a density of vapor emanating from the nebulizing mesh, a flow sensor for assessing an amount of vapor inhaled by a user through the exit port, a flow sensor for assessing an amount of the medicament withdrawn from the cartridge, a position sensor for assessing a change in position of a float or plunger associated with a volume of the medicament, and a pH sensor for assessing a pH level of vapor emanating from the nebulizing mesh.

23: The nebulizer of claim 18, wherein the cartridge includes more than one medicament reservoir each configured to store a medicament.

24. (canceled)

25: The nebulizer of claim 18, wherein the logic module is configured to control power provided to the nebulizing mesh from the power source in response to calibration data received from the remote software module.

26: The nebulizer of claim 21, wherein the logic module is configured to control power provided to the nebulizing mesh from the power source in response to calibration data received from the remote software module and in response to information obtained from the one or more sensors.

27: The nebulizer of claim 25, wherein the logic module is configured to cause the hardware security module to prevent power from flowing from the power source to the nebulizing mesh in response to security information received from the remote software module.

28: The nebulizer of claim 27, wherein the security information includes information associated with an authorized frequency of medicament dispensation.

29: The nebulizer of claim 21, wherein the logic module is configured to receive information obtained from the at least one sensor.

30. (canceled)

31: A method of securely dispensing a medicament to a user, the method comprising:
specifying, in a remote software module, a treatment regimen for a user comprising one or more initial conditions of authorized dispensation of a medicament for the user;
transmitting the treatment regimen information to a hardware device, wherein the hardware device comprises:
a logic module,
a power source in operative communication with the logic module and configured to provide power to a medicament delivery actuator,
a hardware security module in operative communication with the logic module and configured to prevent unauthorized use of the hardware device,
a cartridge comprising the medicament at a first concentration,
a medicament vehicle module in fluid communication with the cartridge and including a medicament vehicle composition, and
an input module in operative communication with the logic module and configured to detect a desired dispensation of the medicament when activated by the user; and
permitting dispensation of the medicament to the user if and only if the activation of the input module by the user satisfies all initial conditions of authorized dispensation.

32: The method of claim 31, wherein the initial conditions of authorized dispensation comprise one or more of: a medicament name, a medicament dosage level, and a medicament dosage frequency.

33: The method of claim 31, wherein the step of permitting dispensation of the medicament comprises enabling, via the logic module, power to flow from the power source to the medicament delivery actuator upon activation of the input module by the user.

34: The method of claim 31 further comprising specifying, in the remote software module, a second treatment regimen for the user comprising one or more modified conditions of authorized dispensation of the medicament for the user that differs from the initial conditions of authorized dispensation.

35: The method of claim 34, wherein the second treatment regimen is specified by a clinician.

36: The method of claim 34, wherein the second treatment regimen is specified by an artificial intelligence or machine learning module associated with the remote software module.

37: The method of claim 36, wherein the artificial intelligence or machine learning module specifies the second treatment regimen based at least in part on an observed improvement in one or more characteristics of the user after an initial period of time.

38: The method of claim 36, wherein the second treatment regimen includes a test condition selected from a list of permitted modified conditions of authorized dispensation.

39: The method of claim 38, wherein the list of permitted modified conditions of authorized dispensation includes one or more of: a modified medicament dosage level, a modified medicament dosage frequency, a modified permitted medicament dispensation time of day, and a modified permitted medicament dispensation time relative to a time of consumption by the user of a second medicament or a meal.

40. (canceled)

41. (canceled)
